(19) United States
(12) Patent Application Publication
(10) Pub. No.: US 2024/0066116 A1
(43) Pub. Date: Feb. 29, 2024

(54) FULLY SYNTHETIC, LONG-CHAIN NUCLEIC ACID FOR VACCINE PRODUCTION TO PROTECT AGAINST CORONAVIRUSES

(71) Applicant: ROCKETVAX AG, Basel (CH)

(72) Inventors: Matthias CHRISTEN, Reinach (CH); Natasa CMILJANOVIC VRANIC, Basel (CH); Vladimir CMILJANOVIC, Giebenach (CH)

(73) Assignee: ROCKETVAX AG, Basel (CH)

(21) Appl. No.: 18/280,150

(22) PCT Filed: Sep. 9, 2021
(86) PCT No.: PCT/EP2021/074738

§ 371 Date: Sep. 1, 2023

(30) Foreign Application Priority Data
Mar. 3, 2021 (WO) PCT/EP2021/055401

(51) Int. Cl. A61K 39/215; C07K 14/005; C12N 7/00; C12N 15/70

(52) U.S. Cl. CPC A61K 39/215; C07K 14/005; C12N 7/00; C12N 15/70; C12N 2770/20022; C12N 2770/20034; C12N 2770/20052; C12N 2800/101

(57) ABSTRACT

This invention describes a fully synthetic, long-chain nucleic acid that can be used in biotechnological manufacturing processes to produce envelope proteins, virus envelopes and fragments of virus envelopes of SARS-CoV-2 and related coronaviruses in highly purified form, which, as a vaccine protect against COVID-19 and other viral diseases

The present invention relates to a fully synthetic, long-chain nucleic acid according to the independent claim 1. The invention further relates to a kit comprising two or more of these nucleic acids and a biotechnological production unit comprising at least one plasmid comprising the nucleic acid. The invention further relates to a virus envelope, a fragment of a virus envelope and/or a virus envelope protein obtainable by gene expression using said nucleic acid. Furthermore, the invention concerns a vaccine comprising products obtainable by gene expression using the nucleic acid, in particular a vaccine against the coronavirus SARS-CoV-2, as well as a method for producing the vaccine.

The rapid development and availability of vaccines is crucial in combating many viruses and bacteria. The production of suitable vaccines is a multi-stage, complex process and is not always successful despite often high investments. Typically, the development of a suitable vaccine takes years. These long development times consist of a major problem, especially with regard to new emerging pathogens, or mutated pathogens, as from an epidemiological point of view it is only possible to react too late, if at all, to the emergence of new diseases. In contrast, the analysis, identification and further detection of new or heavily mutated pathogens are now possible within weeks or even days, which is a huge improvement over the last century.

In this context, viruses are of special interest, as they harbor high mutation rates causing the spread from other species to humans. Rapid spreading of these viruses makes them a major challenge for modern medicine. The usual time today (2020) between the detection/identification of a newly emerging virus and the development of a vaccine is typically years. In a few cases, with sufficient prior knowledge, experimental vaccines could be provided within months. However, this time span is much longer than the typical time until thousands or millions of people are infected. Such rapid spread is also a direct consequence of the high mobility of today's society.

Ideally, immediately after the identification of a new virus, a vaccine would be available in sufficient quantity and of the highest quality and would allow for a nationwide vaccination of all persons who have somehow come close to the initial outbreak site of the new virus. Furthermore, an ideal method for such a vaccine would be capable of reacting to the evolution and adaptation of the virus. Such an ideal production possibility seems utopian to the person skilled in the art today.

In the recent past in particular, the corona pandemic has dramatically increased the relevance of developing suitable tools for vaccine production. There is unanimous agreement that the development of a vaccine against the coronavirus SARS-CoV-2 is the only proven means of containing the pandemic and the associated global crisis in the long term.

In this background, the task of the present invention is to provide an instrument which allows the production of a vaccine against the coronavirus SARS-CoV-2, in large quantities and of high quality.

The problem is solved by a fully synthetic, long-chain nucleic acid according to claim 1. Preferred embodiments of the invention are reflected in the embodiments and dependent claims.

Accordingly, the invention relates to, inter alia, the following embodiments:
1. Fully synthetic, long-chain nucleic acid with at least 4,000 bases, characterised in that the nucleic acid
   comprises
   a) at least two of the four sequence parts A-D in any arrangement, wherein i) Sequence part A comprises
   a) a sequence as defined in SEQ. ID. 50 or a sequence having at least 98.5% sequence identity to the sequence as defined in SEQ. ID. 50; or
   b) a sequence as defined in SEQ. ID. 3 or a sequence having at least 90% sequence identity to the sequence as defined in SEQ. ID. 3;
   ii) Sequence part B comprises
   a) a sequence as defined in SEQ. ID. 48 or a sequence having at least 98.3% sequence identity to the sequence as defined in SEQ. ID. 48; or
   b) a sequence as defined in SEQ. ID. 7 or a sequence having at least 90% sequence identity to the sequence as defined in SEQ. ID. 7;
   iii) Sequence part C comprises
   a) a sequence as defined in SEQ. ID. 49 or a sequence having at least 97.2% sequence identity to the sequence as defined in SEQ. ID. 49; or
   b) a sequence as defined in SEQ. ID. 11 or a sequence having at least 90% sequence identity to the sequence as defined in SEQ. ID. 11;
   iv) Sequence part D comprises a sequence as defined in SEQ. ID. 17 or a sequence having at least 98.5% sequence identity to the sequence as defined in SEQ. ID. 17; or encompasses a ribonucleic acid sequence corresponding to the deoxyribonucleic acid sequence according to the sequence parts A-D; and
   b) does not comprise
   1.) a nucleic acid sequence part encodes an amino acid sequence having the function of a SARS-CoV-2 amino acid sequence encoded by ORF7a; and/or
   2.) a nucleic acid sequence part encodes an amino acid sequence having the function of a SARS-CoV-2 amino acid sequence encoded by ORF3a.
2. The nucleic acid according to embodiment 1, characterized in that it has at least 8′000 bases, preferably at least 20′000 bases, in a defined sequence.
3. The nucleic acid according embodiment 1 or 2, characterized in that the nucleic acid comprises not more than one or no ORF-associated nucleic acid sequence parts, wherein the ORF-associated nucleic acid sequence part encodes an amino acid sequence having the function of a SARS-CoV-2 amino acid sequence encoded by ORF6 or ORF8.
4. The nucleic acid according to embodiment 3, wherein the nucleic acid comprises no ORF-associated nucleic acid sequence part, wherein the ORF-associated nucleic acid sequence part encodes an amino acid sequence having the function of a SARS-CoV-2 amino acid sequence encoded by ORF6 or ORF8.
5. The nucleic acid according to one of the preceding embodiments, wherein the nucleic acid additionally comprises
   a) 1.) an ORF1ab sequence defined by the SEQ. ID. 51 or a sequence having at least 98,5% sequence identity to SEQ. ID. 51; or
   2.) i) an ORF1b sequence defined by the SEQ. ID. 59 or a sequence having at least 98,5% sequence identity to SEQ. ID. 59; and
   ii) a n ORF1a sequence defined by the SEQ. ID. 58 or a sequence having at least 98,6% sequence identity to SEQ. ID. 58; and
   b) an ORF3a sequence defined by the SEQ. ID. 52 or a sequence having at least 99% sequence identity to SEQ. ID 52.
6. The nucleic acid according to embodiment 7, wherein the nucleic acid additionally comprises
   a) an ORF6 sequence defined by the SEQ. ID. 53 or a sequence having at least 94,1% sequence identity to SEQ. ID 53; and/or
   b) an ORF8 sequence defined by the SEQ. ID. 55 or a sequence having at least 99% sequence identity to SEQ. ID 55.
7. The nucleic acid according to one of the preceding embodiments, characterized in that sequence parts A to C correspond to the sequence according to SEQ. ID. 19 or the corresponding ribonucleic acid sequence.
8. The nucleic acid according to any of the preceding embodiments, characterized in that the nucleic acid comprises in any arrangement at least three of the four sequence parts A-D or at least three of four sequence parts with a ribonucleic acid sequence corresponding to the deoxyribonucleic acid sequence according to the sequence parts A-D.
9. The nucleic acid according to any of the preceding embodiments, characterized in that the nucleic acid comprises in any arrangement the four sequence parts A-D or four sequence parts with a ribonucleic acid sequence corresponding to the deoxyribonucleic acid sequence according to the sequence parts A-D.
10. The nucleic acid according to any one of embodiments 1 to 6, characterized in that the nucleic acid comprises two or three of the four sequence parts A-D.
11. The nucleic acid according to embodiment 10, characterized in that the nucleic acid comprises three of the four sequence parts A-D.
12. The nucleic acid according to one of the preceding embodiments, characterized in that the nucleic acid additionally comprises at least one sequence consisting of
   SEQ. ID. 15
   SEQ. ID. 28
   SEQ. ID. 29 and
   SEQ. ID. 30
   or comprises one of the deoxyribonucleic acid sequences according to the sequence parts SEQ. ID. 15, SEQ. ID. 28, SEQ. ID. 29 and SEQ. ID. 30 or the corresponding ribonucleic acid sequence.
13. The nucleic acid according to one of the preceding embodiments, characterized in that it has a maximum size of 1′000′000 bases, preferably a maximum size of 200′000 bases.
14. A vector comprising the nucleic acid according to one of the preceding embodiments.
15. The vector according to embodiment 14, wherein the vector comprises the sequences defined by the SEQ. ID. 46 and SEQ. ID. 47.
16. The vector according to any one of the embodiments 14 to 15, wherein the vector is a plasm id vector.
17. A kit comprising two or more nucleic acids according to one of embodiments 1 to 13.
18. The kit according to embodiment 17, wherein the nucleic acids are present in at least one plasmid, preferably in two or more plasm ids.
19. A biotechnological production unit comprising at least one vector according to embodiments 14 to 16.
20. A virus envelope, a fragment of a virus envelope and/or virus envelope protein obtainable by gene expression using at least one nucleic acid according to one of embodiments 1 to 3, using the vector according to one of embodiments 14 to 16, using the kit according to one of embodiments 17 or 18, or the biotechnological production unit according to embodiment 19, wherein the virus envelope, the fragment of a virus envelope and/or the virus envelope protein package the at least one nucleic acid according to one of embodiments 1 to 13.
21. A vaccine against the coronavirus SARS-CoV-2 comprising at least one nucleic acid according to one of embodiments 1 to 13 and products obtainable by gene expression using at least one nucleic acid according to one of the embodiments 1 to 13, using the vector according to one of embodiments 14 to 16, using the kit according to one of the embodiments 17 or 18 in a production organism, in particular comprising the virus envelope, the fragment of a virus envelope and/or the virus envelope protein according to embodiment 20.
22. The vaccine according to embodiment 21 comprising at least two molecularly precisely defined protein components selected from the group consisting of the protein components a, b1, b2, c1, c2, d1 or d2, wherein
   (i) the protein component a comprises
   a) the sequence according to SEQ. ID. 14 analogous to the S protein of SARS-CoV-2 or a sequence having at least 90% sequence identity to SEQ. ID. 14; or b) the sequence according to SEQ. ID. 18 analogous to the S protein of SARS-CoV-2 or sequence having at least 90% sequence identity to SEQ. ID.18;
   (ii) the protein component b1 comprises
   a) the sequence according to SEQ. ID. 6 analogous to the envelope protein E of SARS-CoV-2 or a sequence having at least 90% sequence identity to SEQ. ID.6; or
   b) the sequence according to SEQ. ID. 21 analogous to the envelope protein E of SARS-CoV-2 or a sequence having at least 90% sequence identity to SEQ. ID.21; and
   the protein component b2 comprises the sequence according to SEQ. ID. 8 analogous to the envelope protein E of MHV59A or an equivalent protein comprising a sequence having at least 90% sequence identity to SEQ. ID.8;
   (iii) the protein component c1 comprises
   a) the sequence according to SEQ. ID. 10 analogous to the envelope protein M of SARS-CoV-2 or a sequence having at least 90% sequence identity to SEQ. ID. 10; or
   b) the sequence according to SEQ. ID. 22 analogous to the membrane protein M of SARS-CoV-2 or a sequence having at least 90% sequence identity to SEQ. ID.22; and
   the protein component c2 comprises the sequence according to SEQ. ID. 12 analogous to membrane protein M of MHV59A or an equivalent protein comprising a sequence having at least 90% sequence identity to SEQ. ID. 12; and
   (iv) the protein component d1 comprises
   a) the sequence according to SEQ. ID. 2 analogous to the nucleocapsid phosphoprotein N of SARS-CoV-2 or a sequence having at least 90% sequence identity to SEQ. ID. 2; or
   b) the sequence according to SEQ. ID. 26 analogous to the nucleocapsid phosphoprotein N of SARS-CoV-2 or a sequence having at least 90% sequence identity to SEQ. ID. 26; and the protein component d2 the sequence according to SEQ. ID. 4 analogous to the nucleocapsid phosphoprotein N of MHV59A or an equivalent protein comprising a sequence having at least 90% sequence identity to SEQ. ID. 4.
23. A method for the production of the vaccine a vaccine against the coronavirus SARS-CoV-2 comprising the successive steps of
   a) introducing the nucleotide acid sequence according to one of embodiments 1 to 13 into a biotechnological production unit, in particular a cell line, wherein, the nucleic acid-based mRNA coding for at least two of the protein components selected from the group consisting of the protein components a, b1, b2, c1, c2, d1 or d2 are prepared by translation;
   b) obtaining protein components from the biotechnological production unit in step a); and
   c) purifying the obtained protein components to obtain the vaccine against the coronavirus SARS-CoV-2.
24. A method for the production of a vaccine against the coronavirus SARS-CoV-2 comprising the virus envelope, the fragment of a virus envelope and/or the virus envelope protein according to embodiment 20 comprising the successive steps of:
   a) introducing the nucleotide acid sequence according to one of embodiments 1 to 13 into a biotechnological production unit, wherein the biotechnological production unit comprises a nucleotide acid coding for at least one of the protein components selected from the group consisting of the protein components a, b1, c1, and d1.
   b) obtaining a fragment of a virus envelope and/or virus envelope protein from the biotechnological production unit in step a); and
   c) purifying the obtained protein components to obtain the vaccine against the coronavirus SARS-CoV-2 comprising the virus envelope, the fragment of a virus envelope and/or the virus envelope protein according to embodiment 20.
25. A method for the production of a vaccine against the coronavirus SARS-CoV-2 comprising the successive steps of:
   a) introducing the vector according to one of embodiments 14 to 16 into an amplifying biotechnological production unit;
   b) amplifying the nucleotide acid according to one of embodiments 1 to 13 in the amplifying biotechnological production unit;
   c) obtaining the nucleotide acid amplified in step b);
   d) obtaining the vaccine against the coronavirus SARS-CoV-2 by using method according to embodiment 23 or 24.

The invention, therefore, relates to a fully synthetic, long-chain nucleic acid with at least 4,000 bases, characterised in that the nucleic acid comprises at least two of the four sequence parts A-D in any arrangement, wherein i) Sequence part A comprises a) a sequence as defined in SEQ. ID. 1 or a sequence having at least 98.5% sequence identity to the sequence as defined in SEQ. ID. 1; or b) a sequence as defined in SEQ. ID. 3 or a sequence having at least 90% sequence identity to the sequence as defined in SEQ. ID. 3; ii) Sequence part B comprises a) a sequence as defined in SEQ. ID. 5 or a sequence having at least 98.3% sequence identity to the sequence as defined in SEQ. ID. 5; or b) a sequence as defined in SEQ. ID. 7 or a sequence having at least 90% sequence identity to the sequence as defined in SEQ. ID. 7; iii) Sequence part C comprises a) a sequence as defined in SEQ. ID. 9 or a sequence having at least 97.2% sequence identity to the sequence as defined in SEQ. ID. 9; or b) a sequence as defined in SEQ. ID. 11 or a sequence having at least 90% sequence identity to the sequence as defined in SEQ. ID. 11; iv) Sequence part D comprises a sequence as defined in SEQ. ID. 13 or a sequence having at least 98.5% sequence identity to the sequence as defined in SEQ. ID. 13; or encompasses a ribonucleic acid sequence corresponding to the deoxyribonucleic acid sequence according to the sequence parts A-D.

In certain embodiments, the invention relates to a fully synthetic, long-chain nucleic acid with at least 4,000 bases, characterised in that the nucleic acid comprises a) at least two of the four sequence parts A-D in any arrangement, wherein i) Sequence part A comprises a sequence as defined in SEQ. ID. 50 or a sequence having at least 98.5% sequence identity to the sequence as defined in SEQ. ID. 50; ii) Sequence part B comprises a sequence as defined in SEQ. ID. 48 or a sequence having at least 98.3% sequence identity to the sequence as defined in SEQ. ID. 48;iii) Sequence part C comprises a sequence as defined in SEQ. ID. 49 or a sequence having at least 97.2% sequence identity to the sequence as defined in SEQ. ID. 49; iv) Sequence part D comprises a sequence as defined in SEQ. ID. 17 or a sequence having at least 98.5% sequence identity to the sequence as defined in SEQ. ID. 17; or encompasses a ribonucleic acid sequence corresponding to the deoxyribonucleic acid sequence according to the sequence parts A-D; and b) 1.) a nucleic acid sequence part encodes an amino acid sequence having the function of a SARS-CoV-2 amino acid sequence encoded by ORF7a; and/or 2.) a nucleic acid sequence part encodes an amino acid sequence having the function of a SARS-CoV-2 amino acid sequence encoded by ORF3a.

The nucleic acid according to the invention allows to significantly accelerate the production of the mentioned vaccines and leads to well-defined vaccines which are very specific to a virus or a modification, especially to the coronavirus SARS-CoV-2.

As will be shown further below, specific sequence features, of the sequence parts comprised in the nucleic acid sequence, according to the invention, allow the nucleic acid to be produced fully synthetically and thus tailor-made. The nucleic acid, according to the invention, thus differs from the nucleic acid naturally present in coronaviruses, not only in that it is in certain embodiments DNA instead of RNA, but also in the sequence which, in contrast to the naturally occurring sequence, allows the fully synthetic production of the nucleic acid by means of chemical synthesis.

Ultimately, the nucleic acid according to the invention thus makes it possible to express protein components that are defined with molecular precision. When these protein components are administered as a vaccine, optimal immunization can thus be obtained in the vaccine recipient. At the same time, the risk of possible side effects, which is highly prevalent with imprecisely defined protein components, is greatly minimized. Also, the fact that the protein components can be produced using common expression systems used for protein expression means that vaccines can be made available in large quantities very quickly. This is of crucial importance for viruses such as the coronavirus SARS-Cov-2, whose spread has assumed the proportions of a pandemic and whose containment, therefore, requires widespread vaccine administration.

The following terms and concepts shall be used in the context of this invention:

The term “nucleic acid”, refers to either DNA, RNA, and any modifications thereof. The nucleic acid may be single-stranded or double-stranded. Modifications include, but are not limited to, those which provide other chemical groups that incorporate additional charge, polarizability, hydrogen bonding, electrostatic interaction, and fluxionality to the nucleic acid ligand bases or the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine. Modifications can also include 3′ and 5′ modifications such as capping.

Fully synthetic. From a chemical point of view, nucleic acids are very sophisticated molecules with repeating units, the so-called bases. The term “fully synthetic” in this context means that the nucleic acid according to the invention is produced by a series of chemical reaction steps using chemical reagents. Biochemical aids such as enzymes can also be used during individual late production steps, such as the joining of already longer oligomers. The latter can in turn optionally be synthetic. Fully synthetic nucleic acids have sequence features that enable the chemical production process and differ from naturally occurring nucleic acids in one or more of the following sequence features:
i) the absence of one or more enzymatic restriction sites, in particular, restriction sites for Type IIS restriction endonucleases, which are known to the person skilled in the art;
ii) the absence or reduced occurrence, compared to the corresponding naturally occurring nucleic acids, of repeating nucleic acid sequences with more than 9 consecutive units of the same base within the fully synthetic nucleic acid;
iii) the absence or reduced occurrence of repeating base-pair sequences with more than 12 bases compared to the corresponding naturally occurring nucleic acids;
iv) the absence or reduced occurrence, relative to the corresponding naturally occurring nucleic acids, of indirectly repeating base-pair segments consisting of more than 12 base units known to the person skilled in the art as reverse-complementary sequences therefor;
v) the absence or reduced occurrence, relative to the corresponding naturally occurring nucleic acids, of nucleic acid sequences with more than 9 consecutive repetitions of duplicate base units (dinucleotide repeats) known to the person skilled in the art; and
vi) the absence or reduced occurrence, relative to the corresponding naturally occurring nucleic acids, of nucleic acid sequences with more than five consecutive repetitions of triple base units (trinucleotide repeats) known to the person skilled in the art.

In some embodiments, fully synthetic nucleic acids are partly produced and/or comprise sequence features according to the methods described in Venetz, J. E., et al., 2019, Proceedings of the National Academy of Sciences, 116(16), 8070-8079 and/or the SI appendix thereof.

In some embodiments, fully synthetic nucleic acids comprise sequence features that enable the chemical production process and differ from naturally occurring nucleic acids in two or more of the above-mentioned sequence features, in particular of the above-mentioned sequence features i)-vi).

In some embodiments, fully synthetic nucleic acids comprise sequence features that enable the chemical production process and differ from naturally occurring nucleic acids in three or more of the above-mentioned sequence features, in particular of the above-mentioned sequence features i)-vi).

In some embodiments, fully synthetic nucleic acids comprise sequence features that enable the chemical production process and differ from naturally occurring nucleic acids in four or more of the above-mentioned sequence features, in particular of the above-mentioned sequence features i)-vi).

In some embodiments, fully synthetic nucleic acids comprise sequence features that enable the chemical production process and differ from naturally occurring nucleic acids in five or more of the above-mentioned sequence features, in particular of the above-mentioned sequence features i)-vi).

In some embodiments, fully synthetic nucleic acids comprise sequence features that enable the chemical production process and differ from naturally occurring nucleic acids in six of the above-mentioned sequence features, in particular of the above-mentioned sequence features i)-vi). Long-chain oligonucleotides have been commercially available for years in short fragments, typically producing pieces with 60, 100 or 200 bases. Massively longer oligonucleotides are not readily available because the syntheses used today have too high error rates to produce reasonable amounts of longer nucleic acids. Such fragments with less than 1000 bases are, therefore, called short-chain, while nucleic acids with 1000 bases or more are called long-chain. Long-chain nucleic acids with 1000 to 5000 bases can be produced today at considerable expense (e.g. by the companies Twist Bioscience, Life-technologies). Long-chain nucleic acids with more than 5000 bases are enormously complex but chemically well-defined molecules. Each of the molecules can be described completely in terms of classical organic chemistry by position, type and linkage with other parts of the molecule. Two identical long-chain nucleic acids are therefore, despite their size and despite the fact that they contain tens of thousands to millions of atoms, identical in that all components are identical and are identically linked.

An explanation of end groups, any residues of protective groups or other auxiliaries from the synthesis of nucleic acids. The above description refers to the type of base in the nucleic acid. The person skilled in the art is aware that the synthesis is carried out by means of various auxiliary agents which are cleaved off at the end. However, sometimes residues of such groups remain, or other parts of the molecule are derivatised before or after a synthesis step. Such groups are known to the person skilled in the art and include, among others, Poly-A tail, modified DNA bases, cleavable linkers from solid-phase synthesis, biochemical groups such as biotin or streptavidin and others.

Other possible modifications and modifications that are used in standard methods concern fluorescent markers. These modifications or residues thereof should not affect the above description, and a group of identical nucleic acids should be considered identical if all n bases at their positions per position and type base are identical for all bases. In other words, the nucleic acids of the invention also include nucleic acids with the above modifications or residues, provided they have the base sequences required by the invention.

According to a first aspect, the present invention thus concerns nucleic acids which have special properties. These special properties are comprised in the base sequence, i.e. the sequence, and are only obtained if the inventive nucleic acid has certain properties. These properties are directly parts of the specific molecule or the entire chemically comprehensive description of the specific molecule. However, for the sake of simplicity, the base sequence should be indicated within this description in the text, always making it clear that the specific molecule is meant. The base sequence is therefore only a pragmatic form of description and obviously better suited for a textual representation of the invention than a direct representation of the molecule, or its IUPAC name.

The inventive molecules acquire the special properties through the presence of specific sequences, analogous to a description of a group of classical chemical agents with one or more molecular parts, which in chemistry are typically abbreviated as “R” and can then be described in more detail by describing “R”. Thus, in the present invention, analogous to this usual procedure in organic chemistry, a group of sequences is described which are responsible for the special properties of the inventive long-chain fully synthetic nucleic acids.

The inventive nucleic acids are characterized by the fact that they comprise fully synthetic nucleic acids which code for at least two of the 4 types of proteins of envelope proteins coronaviruses.

The term “types of envelope proteins of coronaviruses”, as used herein, refers to group A, group B, group C, or group D proteins of a coronavirus. The term “group A” protein, as used herein, refers to the group of nucleocapsid protein (N-type) of coronavirus. The term “group B”, as used herein, refers to the group of envelope proteins (E-type) of coronaviruses. The term “group C” protein, as used herein, refers to the membrane protein (M-type) of coronavirus. The term “group D” protein, as used herein, refers to the glycosylated surface protein (S-type) of coronavirus.

In some embodiments, the nucleic acids described herein are characterized by the fact that they comprise nucleic acids which code for at least one group A protein and at least one group B protein. In some embodiments, the nucleic acids described herein are characterized by the fact that they comprise nucleic acids which code for at least one group A protein and at least one group C protein. In some embodiments, the nucleic acids described herein are characterized by the fact that they comprise nucleic acids which code for at least one group A protein and at least one group D protein. In some embodiments, the nucleic acids described herein are characterized by the fact that they comprise nucleic acids which code for at least one group B protein and at least one group C protein. In some embodiments, the nucleic acids described herein are characterized by the fact that they comprise nucleic acids which code for at least one group B protein and at least one group D protein. In some embodiments, the nucleic acids described herein are characterized by the fact that they comprise nucleic acids which code for at least one group C protein and at least one group D protein.

In some embodiments, the inventive nucleic acids are characterized by the fact that they
(a) comprise more than 4,000 bases in a well-defined sequence; and
(b) comprise at least 2 of 4 sequences of particular importance which are assigned to the 4 sequence groups A-D which code for the 4 types of envelope proteins of coronaviruses, wherein
   i) the first sequence group A encodes for envelope proteins of the nucleocapsid protein N of coronavirus,
   ii) the second sequence group B encodes for envelope proteins of the type envelope protein E of coronaviruses,
   iii) the third sequence group C encodes for envelope proteins of the membrane protein M type of coronavirus, and
   iv) the fourth sequence group D encodes for envelope proteins of the glycosylated surface protein S of coronavirus.

The sequence part A disclosed within this description comprises a sequence according to SEQ. ID. 1 or SEQ. ID. 3, coding for the corresponding protein sequences according to SEQ. ID. 2 or SEQ. ID. 4. In some embodiments, the sequence part A comprises the sequence defined by the SEQ. ID. 50 or sequence having at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 50.

In some embodiments, the sequence part A comprises a sequence having at least 90% sequence identity to SEQ. ID. 3.

In some embodiments, the sequence part A comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 2.

In some embodiments, the sequence part A comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 4.

In some embodiments, the sequence part A comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 2 and SEQ. ID. 4.

The sequence part B disclosed within this description comprises a sequence according to SEQ. ID. 5 or SEQ. ID. 7, coding for the corresponding protein sequences according to SEQ. ID. 6 or SEQ. ID. 8. In some embodiments, the sequence part B comprises the sequence defined by the SEQ. ID 48 or a sequence having at least 98.3%, at least 98.6%, at least 99.1%, or at least 99.5% sequence identity to SEQ. ID. 48.

In some embodiments, the sequence part B comprises a sequence having at least 90% sequence identity to SEQ. ID. 7.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 6.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 8.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% SEQ. ID. 6 and SEQ. ID. 8.

The sequence part C disclosed within this description comprises a sequence according to SEQ. ID. 9 or SEQ. ID. 11, coding for the corresponding protein sequences according to SEQ. ID. 10 or SEQ. ID. 12. In some embodiments, the sequence part C comprises the sequence defined by the SEQ. ID. 49 or a sequence having at least 97.2%, at least 97.4%, at least 97.6%, at least 97.8%, at least 98%, at least 98.2%, at least 98.4%, at least 98.6%, at least 98.8%, at least 99%, at least 99.2%, at least 99.4%, at least 99.6%, at least 99.8%, at sequence identity to SEQ. ID. 49.

In some embodiments, the sequence part C comprises a sequence having at least 90% sequence identity to SEQ. ID. 11.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to sequence identity to SEQ. ID. 12.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 10.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 10 and SEQ. ID. 12.

The sequence part D disclosed within this description comprises a sequence according to SEQ. ID. 13, coding for the corresponding protein sequence according to SEQ. ID. 14. In some embodiments, the sequence part D comprises the sequence defined by the SEQ. ID 17 or a sequence having at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 17.

In some embodiments, the sequence part B comprises a sequence coding for an amino acid sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 14.

The term “percent (%) sequence identity” with respect to a reference sequence is defined as the percentage of nucleotides or amino acid residues in a candidate sequence that are identical with the nucleotides or amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In some embodiments, the nucleotide acid sequence of the invention is altered (e.g., to facilitate the production process of the nucleotide acid sequence or products thereof) without altering or by unsubstantially altering the properties of the protein products.

In some embodiments, the alterations of the nucleotide acid sequence of the invention include at least one alteration selected from the group of
1) base substitutions insertions, or deletions relative to the reference sequence without altering or by unsubstantially altering the properties of the protein products;
2) replacing codons with synonymous versions; and
3) reduction of the number of hypothetical genetic elements present within protein-coding sequences such as (alternative) ORFs, predicted gene internal transcriptional start sites, and/or sequence motifs (predicted or cryptic) that fine-tune translation rates (e.g., ribosome stalling motifs).

Testing whether the genes of the altered nucleotide acid sequence of the invention remain functional will identify genes in which additional information beyond the amino acid code is necessary for proper functioning.

In some embodiments, the nucleotide acid sequence described herein is altered to improve the biological function of the encoded protein products.

Such a biological function includes but is not limited to stability enhancement, production facilitation (e.g., insertion of additional replication initiating sequences), replication limitation.

In some embodiments, the nucleotide acid sequence described herein is altered to encode at least one alternative protein of interest with a similar structure but alternative biological function, such as the function of a protein of a mutated virus.

The person skilled in the art can obtain such an altered nucleotide sequence by analyzing the sequence coding for at least one alternative protein of interest (e.g. the nucleotide acid sequence of a mutated virus) and implementing the relevant alterations (e.g. mutations) into the most similar nucleotide acid sequence described herein. In some embodiments, the nucleotide acid sequences the most similar nucleotide acid sequence described herein is a sequence defined by the SEQ. ID. 1, SEQ. ID. 3, SEQ. ID. 5, SEQ. ID. 7, SEQ. ID. 9, SEQ. ID. 11, SEQ. ID. 13, and/or SEQ. ID. 17.

In some embodiments, the nucleotide acid sequences the most similar nucleotide acid sequence described herein is a sequence defined by the SEQ. ID. 1, SEQ. ID. 5, SEQ. ID. 9, SEQ. ID. 13.

In some embodiments, the coronavirus described herein is SARS-CoV-2. In some embodiment the SARS-CoV-2 described herein is a SARS-CoV-2 variant selected from the group of Lineage B.1.1.207, Lineage B.1.1.7, Cluster 5, 501.V2 variant, Lineage P.1, Lineage B.1.429/CAL.20C, and Lineage B.1.525.

In some embodiments, the SARS-CoV-2 described herein is a SARS-CoV-2 variant described by a Nextstrain Glade selected from the group 19A, 20A, 20C, 20G, 20H, 20B, 20D, 20F, 20I, and 20E.

In some embodiments, the sequence coding for at least one alternative protein of interest comprises sequences coding for a protein that is characteristic for at least one SARS-CoV-2 variant. In some embodiments, the protein that is characteristic for at least one SARS-CoV-2 variant is a protein that is encoded by a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequences SEQ. ID. 18, SEQ. ID. 21, SEQ. ID. 22 and/or SEQ. ID. 26.

This implementation of the relevant alterations can be achieved for example by insertion, deletion, substitution, and/or modification of at least one base, but not more than the percentage of the nucleotide acid sequence described herein.

In some embodiments, the nucleotide acid sequences the most similar nucleotide acid sequence described herein is a sequence defined by the SEQ. ID. 1, SEQ. ID. 3, SEQ. ID. 5, SEQ. ID. 7, SEQ. ID. 9, SEQ. ID. 11, SEQ. ID. 13, and/or SEQ. ID. 17.

In some embodiments, the nucleotide acid sequences the most similar nucleotide acid sequence described herein is at least one sequence defined by the SEQ. ID. 1, SEQ. ID. 5, SEQ. ID. 9, and/or SEQ. ID. 13.

In some embodiments, the insertion, deletion, or modification can be achieved by de novo synthesis of the nucleic acid of the invention using a series of chemical reaction steps using chemical reagents as described herein.

Altered sequences may comprise sequence features (e.g., the sequence features i)-vi) described above) that enable and/or improve the chemical production process of the altered sequences at more or different positions than the nucleotide acid sequences defined by the SEQ. ID. 1, SEQ. ID. 3, SEQ. ID. 5, SEQ. ID. 7, SEQ. ID. 9, SEQ. ID. 11, SEQ. ID. 13, and/or SEQ. ID. 17.

Their possible transformation into an IUPAC-classifiable molecule is known to the person skilled in the art. As an alternative to the deoxyribonucleic acid as defined above, a corresponding ribonucleic acid may also be present. In other words, in addition to the deoxyribonucleic acid sequence according to the sequence parts A-D, the definition according to the invention also includes a corresponding ribonucleic acid sequence. In these, the corresponding ribonucleic acid has sequence parts as defined above in which thymine (T) is replaced by uracil (U).

The base-pair sequence of the inventive long-chain nucleic acids, coding for the envelope proteins E, M, N and S of MHV and SARS-CoV-2 and, if applicable, the RNA-dependent RNA polymerase of MHV, represents the result of a complex development, in which, in a first step, a large number of sequence variants were formed by calculation starting from the natural amino acid sequence of the corresponding proteins, considering the redundancy of the genetic code.

Particularly, the base-pair sequence of the inventive long-chain nucleic acids, coding for the proteins E, M, N and/or S of SARS-CoV-2 represents the result of a complex development, in which, in a first step, a large number of sequence variants were formed by calculation starting from the natural amino acid sequence of the corresponding proteins, considering the redundancy of the genetic code.

From the resulting sequence tree, in a second step, the base-pair sequence for each of the encoded envelope proteins was determined which, firstly, is most similar to the natural sequence in terms of biological functionality and, secondly, also has the optimal sequence characteristics to enable the chemical production process.

Further, the sequences encode a combination of structural proteins of the wild-type virus. This enables a broad range of epitopes available to the immune system including T-cell epitopes (see, e.g., Grifoni, A., et al., 2020, Cell, 181(7), 1489-1501). This broad range of epitopes may enable immunity against a broad range of virus variants in patients with or without pre-existing immunity.

Accordingly, the invention is at least in part based on the discovery that the nucleic acid of the invention, enables efficient production of a combination virus-like proteins with limited replication capabilities but similar antigenic effect to the original virus.

As mentioned, the nucleic acid according to the invention has at least 4′000 bases or base-pairs. Preferably, it has at least 8′000 bases, particularly preferably at least 20′000 bases, in a defined sequence. Further, it is preferred that the nucleic acid has a maximum size of 1′000′000 bases, preferably a maximum size of 200′000 bases.

Large sequences have repeatedly shown to be difficult to produce, amplify and/or express, but the large number of bases is beneficial to consistently produce a certain combination of viral-like proteins that have a similar antigenic effect to the original virus.

The means and methods provided herein, enable the production of the nucleic acid according to the invention in a certain length range (see, e.g., Example 1-3).

Accordingly, the invention is at least in part based on the discovery that the nucleic acid of the invention with a length in a certain length range, enables efficient production of a combination virus-like proteins with limited replication capabilities but similar antigenic effect to the original virus.

The nucleic acid according to the invention can be present in the form of a single long-chain nucleic acid or divided into separate long-chain nucleic acids.

In some embodiments, the nucleic acid according to the invention can be present in the form of a single long-chain nucleic acid or divided into up to 4 separate long-chain nucleic acids.

The separation into separate long-chain nucleic acids may facilitate amplification of the nucleic acids of the invention (Example 3).

According to a further preferred embodiment, the sequence parts A-D are arranged according to the sequence SEQ. ID. 16.

It is also preferred that sequence part D consists of SEQ. ID. 17, and codes for the protein sequence according to SEQ. ID. 18.

According to a further preferred embodiment, the sequence parts A-C are arranged according to the sequence SEQ. ID. 19 whereby the sequence part A codes for the protein sequence according to SEQ. ID. 26, the sequence part B codes for the protein sequence according to SEQ. ID. 21, the sequence part C encodes the protein sequence according to SEQ. ID. 22, and in addition the sequence parts A-C can be extended with sequences coding for SEQ. ID. 20, SEQ. ID. 22, SEQ. ID. 23, SEQ. ID. 24, SEQ. ID. 25 and SEQ. ID. 27.

In some embodiments, the invention relates to the nucleotide acid sequence according the invention, wherein the nucleotide acid sequence is defined by a sequence having at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to the SEQ. ID. 19 or the corresponding ribonucleic acid sequence.

It is particularly preferred to supplement the sequence parts A-D disclosed within this description with a nucleic acid sequence of sequence part E, which comprises a sequence according to SEQ. ID. 15 or SEQ. ID. 30, coding for the polyprotein sequences according to SEQ. ID. 31 and SEQ. ID. 32 of the RNA-dependent RNA polymerase of coronavirus.

The sequence according to SEQ. ID. 15 or SEQ. ID. 30 may represent a component of the nucleic acid according to the invention and thus be present in the same molecule in combination with two or more sequences of sequence parts A-D. It is also conceivable that it is present in a kit together with the nucleic acid according to the invention as a component of an independent molecule. The possible transfer into an IUPAC-classifiable molecule is known to the person skilled in the art.

The presence of sequence part E is relevant if, for gene expression of the corresponding proteins, RNA is introduced into a biotechnological production unit instead of a DNA plasmid. In this respect, it is also conceivable that sequence part E is introduced into a kit in the form of RNA according to SEQ. ID. 33 or SEQ. ID. 34 is present in a kit. This will be explained further in the context of the specific examples below.

It has been shown that these concrete sequences are particularly advantageous firstly with regard to their similarity to the natural sequence or their biological functionality and secondly with regard to the chemical production process.

According to another preferred embodiment, the nucleic acid comprises at least three of the four sequence parts A-D in any arrangement. In this respect, it is particularly preferred that the nucleic acid comprises the four sequence parts A-D in any arrangement.

In certain embodiments, the invention relates to the nucleic acid according to the invention, characterized in that the nucleic acid comprises two or three of the four sequence parts A-D.

In certain embodiments, the invention relates to the nucleic acid according to the invention, characterized in that the nucleic acid comprises three of the four sequence parts A-D.

Therefore, in some embodiments, the sequences encode a combination of two or three structural proteins of the wild-type virus or proteins with equivalent functions thereof. This enables a broad range of epitopes available to the immune system including T-cell epitopes (see, e.g., Grifoni, A., et al., 2020, Cell, 181(7), 1489-1501). This broad range of epitopes may enable immunity against a broad range of virus variants in patients with or without pre-existing immunity.

Accordingly, the invention is at least in part based on the discovery that the nucleic acid of the invention, enables efficient production of a combination virus-like proteins with limited replication capabilities but similar antigenic effect to the original virus.

Further, it is preferred that the nucleic acid additionally comprises at least one sequence consisting of the group of
SEQ. ID. 15
SEQ. ID. 28
SEQ. ID. 29 and
SEQ. ID. 30.

In some embodiments, the nucleic acid of the invention comprises one of the deoxyribonucleic acid sequences according to the sequence parts SEQ. ID. 15, SEQ. ID. 28, SEQ. ID. 29 and SEQ. ID. 30 or the corresponding ribonucleic acid sequence.

In some embodiments, the invention relates to the nucleic acid according to the invention, characterized in that the nucleic acid comprises SEQ. ID. 28, or the corresponding ribonucleic acid sequence.

In some embodiments, the invention relates to the nucleic acid according to the invention, characterized in that the nucleic acid comprises SEQ. ID. 29, or the corresponding ribonucleic acid sequence.

In some embodiments, the invention relates to the nucleic acid according to the invention, characterized in that the nucleic acid comprises SEQ. ID. 28 and SEQ. ID. 29, or the corresponding ribonucleic acid sequence.

The nucleic acids of the invention have the special property that they can be incorporated by standard methods into a cell line or other production organism and stimulate the production of fragments or whole envelopes of a virus. The standard methods required for this purpose are known to the person skilled in the art and are described in the context of the concrete examples.

In certain embodiments, the invention relates to the nucleic acid of the invention, characterized in that the nucleic acid comprises not more than one or no ORF-associated nucleic acid sequence parts, wherein each ORF-associated nucleic acid sequence part encodes an amino acid sequence having the function of a SARS-CoV-2 amino acid sequence encoded by ORF6, or ORF8.

In some embodiments, the invention relates to the nucleic acid of the invention, characterized in that the nucleic acid comprises one ORF-associated nucleic acid sequence part, wherein each ORF-associated nucleic acid sequence part encodes an amino acid sequence having the function of a SARS-CoV-2 amino acid sequence encoded by ORF6 or ORF8.

The inventors found, that virus particles can be amplified and subsequently translated and successfully assembled despite omission of certain ORFs that were considered useful for the effective replicability of the original virus. The resulting virus particle may still be able to infect cells and induce the production of non-infective virus fragments.

The inventors found that the ORF6 and ORF8 of the SARS-CoV-2 virusgenome (see FIG. 5) can be omitted, dysfunctional or deleted and virus assembly remains possible.

In certain embodiments, the invention relates to the nucleic acid of the invention, wherein one ORF-associated nucleic acid sequence part encodes an amino acid sequence having the function of a SARS-CoV-2 amino acid sequence encoded by ORF3a.

The phrase “sequence having the function of a SARS-CoV-2 amino acid sequence”, as used herein, refers to a sequence having the function of a SARS-CoV-2 amino acid sequence encoded by the sequence as defined by the SEQ. ID. 60. The structure and function of SARS-CoV-2 amino acid sequences are known in the art (see e.g., Yadav, Rohitash et al., 2021, Cells vol. 10,4 821; Arya, Rimanshee, et al., 2021, Journal of molecular biology 433.2: 166725; Gorkhali, R., et al., 2021, Bioinformatics and Biology Insights, 15, 11779322211025876; Redondo N, et al., 2021, Front Immunol. Jul 7; 12:708264). In some embodiments, the sequence having the function of a SARS-CoV-2 amino acid sequence described herein is a sequence comprised in SEQ. ID. 60 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence comprised in SEQ. ID. 60. Such % sequence variation can for example derive from one or more mutations of a SARS-CoV-2 variant in the SEQ. ID. 60 or from insertions, deletions and/or replacements, preferably conservative insertions, deletions and/or replacements that alter the sequence without altering or without substantially altering the function of the encoded amino acid sequence.

The function of a SARS-CoV-2 amino acid sequence encoded by ORF3a as well as ORF3a sequences and mutations thereof are known in the art (see e.g. Bianchi M, et al., 2021, Int J Biol Macromol. 2021; 170:820-826.) The most common mutations in the ORF3a sequence are V13L, Q57H, Q57H+A99V, G196V and G252V. In some embodiments, the ORF-associated nucleic acid sequence part encoding an amino acid sequence having the function of ORF3a is a sequence encoding SEQ. ID. 20 or a sequence encoding SEQ. ID. 20 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ. ID. 20. In some embodiments, the ORF-associated nucleic acid sequence part encoding an amino acid sequence having the function of ORF3a is a sequence as defined by SEQ. ID. 52 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 52. Such % sequence variation can for example derive from one or more mutations described in Bianchi M, et al., 2021, Int J Biol Macromol. 2021; 170:820-826 or from insertions, deletions and/or replacements, preferably conservative insertions, deletions and/or replacements that alter the sequence without altering or without substantially altering the function of the encoded amino acid sequence.

The function of a SARS-CoV-2 amino acid sequence encoded by ORF6 as well as ORF6 sequences and mutations thereof are known in the art (see e.g. Hassan, Sk Sarif, Pabitra Pal Choudhury, and Bidyut Roy, 2021, Meta Gene 28: 100873.) In some embodiments, the ORF-associated nucleic acid sequence part encoding an amino acid sequence having the function of ORF6 is a sequence encoding SEQ. ID. 23 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ. ID. 23. In some embodiments, the ORF-associated nucleic acid sequence part encoding an amino acid sequence having the function of ORF6 is a sequence as defined by SEQ. ID. 53 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 53. Such % sequence variation can for example derive from one or more mutations described in Hassan, Sk Sarif, Pabitra Pal Choudhury, and Bidyut Roy, 2021, Meta Gene 28: 100873 or from insertions, deletions and/or replacements, preferably conservative insertions, deletions and/or replacements that alter the sequence without altering or without substantially altering the function of the encoded amino acid sequence.

The function of a SARS-CoV-2 amino acid sequence encoded by ORF7a as well as ORF7a sequences and mutations thereof are known in the art (see e.g. Yashvardhini, Niti, et al., 2021, Biomedical Research and Therapy 8.8: 4497-4504.) In some embodiments, the ORF-associated nucleic acid sequence part encoding an amino acid sequence having the function of ORF7a is a sequence encoding SEQ. ID. 24 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ. ID. 24. In some embodiments, the ORF-associated nucleic acid sequence part encoding an amino acid sequence having the function of ORF7a is a sequence as defined by SEQ. ID. 54 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 54. Such % sequence variation can for example derive from one or more mutations described in Yashvardhini, Niti, et al., 2021, Biomedical Research and Therapy 8.8: 4497-4504 or from insertions, deletions and/or replacements, preferably conservative insertions, deletions and/or replacements that alter the sequence without altering or without substantially altering the function of the encoded amino acid sequence. The function of a SARS-CoV-2 amino acid sequence encoded by ORF8 as well as ORF8 sequences and mutations thereof are known in the art (see e.g. Badua, Christian Luke DC, Karol Ann T. Baldo, and Paul Mark B. Medina., 2021, Journal of medical virology 93.3: 1702-1721; Hassan, Sk Sarif, et al., 2021, Computers in biology and medicine 133: 104380.) In some embodiments, the ORF-associated nucleic acid sequence part encoding an amino acid sequence having the function of ORF8 is a sequence encoding SEQ. ID. 25 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the sequence encoding SEQ. ID. 25. In some embodiments, the ORF-associated nucleic acid sequence part encoding an amino acid sequence having the function of ORF8 is a sequence as defined by SEQ. ID. 55 having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 55. Such % sequence variation can for example derive from one or more mutations described in Badua, Christian Luke DC, Karol Ann T. Baldo, and Paul Mark B. Medina., 2021, Journal of medical virology 93.3: 1702-1721 or from insertions, deletions and/or replacements, preferably conservative insertions, deletions and/or replacements that alter the sequence without altering or without substantially altering the function of the encoded amino acid sequence.

The inventors found that the sequences equivalent to the ORF6, ORF7a and ORF8 of the SARS-CoV-2 virusgenome can be omitted, dysfunctional or deleted and virus assembly remains possible.

In some embodiments, the invention relates to the nucleic acid according to the invention, wherein the nucleic acid additionally comprises
a) 1.) an ORF1ab sequence defined by the SEQ. ID. 51 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 51; or
2.) i) an ORF1b sequence defined by the SEQ. ID. 59 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 59; and
ii) an ORF1a sequence defined by the SEQ. ID. 58 or a sequence having at least 98,6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 58;
b) an ORF3a sequence defined by the SEQ. ID. 52 or a sequence having at least 99%, at least 99.1%, at least 99.3%, at least 99.4%, at least 99.5%, at least 99.7%, at least 99.8%, or at least 99.9% sequence identity to SEQ. ID 52; and
c) an ORF7a sequence defined by the SEQ. ID. 54 or a sequence having at least 99,5% sequence identity to SEQ. ID. 54.

In some embodiments, the invention relates to the nucleic acid according to the invention, wherein the nucleic acid additionally comprises
a) 1.) an ORF1ab sequence defined by the SEQ. ID. 51 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 51; or
2.) i) an ORF1b sequence defined by the SEQ. ID. 59 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 59; and
ii) a n ORF1a sequence defined by the SEQ. ID. 58 or a sequence having at least 98,6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 58;
b) an ORF3a sequence defined by the SEQ. ID. 52 or a sequence having at least 99%, at least 99.1%, at least 99.3%, at least 99.4%, at least 99.5%, at least 99.7%, at least 99.8%, or at least 99.9% sequence identity to SEQ. ID 52;
c) an ORF7a sequence defined by the SEQ. ID. 54 or a sequence having at least 99,5% sequence identity to SEQ. ID. 54; and
d) an ORF8 sequence defined by the SEQ. ID. 55 or a sequence having at least 99%, having at least 99.3%, or having at least 99.6% sequence identity to SEQ. ID 55.

In some embodiments, the invention relates to the nucleic acid according to the invention, wherein the nucleic acid additionally comprises
a) 1.) an ORF1ab sequence defined by the SEQ. ID. 51 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 51; or
2.) i) an ORF1b sequence defined by the SEQ. ID. 59 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 59; and
ii) a n ORF1a sequence defined by the SEQ. ID. 58 or a sequence having at least 98,6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 58;
b) an ORF3a sequence defined by the SEQ. ID. 52 or a sequence having at least 99%, at least 99.1%, at least 99.3%, at least 99.4%, at least 99.5%, at least 99.7%, at least 99.8%, or at least 99.9% sequence identity to SEQ. ID 52;
c) an ORF7a sequence defined by the SEQ. ID. 54 or a sequence having at least 99,5% sequence identity to SEQ. ID. 54; and
d) an ORF6 sequence defined by the SEQ. ID. 53 or a sequence having at least 94,1% at least 94.7%, at least 95.2%, at least 95.8%, at least 96.3%, at least 96.8%, at least 97.4%, at least 97.9%, or at least 98.5%, at least 99%, or at least 99.6%, sequence identity to SEQ. ID 53.

In some embodiments, the invention relates to the nucleic acid according to the invention, wherein the nucleic acid additionally comprises
a) 1.) an ORF1ab sequence defined by the SEQ. ID. 51 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 51; or
2.) i) an ORF1b sequence defined by the SEQ. ID. 59 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 59; and
ii) a n ORF1a sequence defined by the SEQ. ID. 58 or a sequence having at least 98,6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 58;
b) an ORF3a sequence defined by the SEQ. ID. 52 or a sequence having at least 99%, at least 99.1%, at least 99.3%, at least 99.4%, at least 99.5%, at least 99.7%, at least 99.8%, or at least 99.9% sequence identity to SEQ. ID 52;
c) an ORF7a sequence defined by the SEQ. ID. 54 or a sequence having at least 99,5% sequence identity to SEQ. ID. 54;
d) an ORF6 sequence defined by the SEQ. ID. 53 or a sequence having at least 94,1% at least 94.7%, at least 95.2%, at least 95.8%, at least 96.3%, at least 96.8%, at least 97.4%, at least 97.9%, at least 98.5%, at least 99%, or at least 99.6%, sequence identity to SEQ. ID 53; and
e) an ORF8 sequence defined by the SEQ. ID. 55 or a sequence having at least 99%, having at least 99.3%, or having at least 99.6% sequence identity to SEQ. ID 55.

In some embodiments, the invention relates to the nucleic acid according to the invention, wherein the nucleic acid additionally comprises a 3′UTR, a 5′UTR, a TRS-L, a TRS-B: S, a TRS-B: orf3a, a TRS-B: E, a TRS-B: M, a TRS-B: orf6, a TRS-B: orf7a, a TRS-B: orf8 and/or a TRS-B: N.

In some embodiments, the invention relates to the nucleic acid according to the invention, wherein the nucleic acid additionally comprises a 3′UTR defined by the SEQ. ID. 57 and/or a 5′UTR defined by the SEQ. ID. 56.

In some embodiments, the invention relates to the nucleic acid according to the invention, wherein the nucleic acid additionally comprises a TRS-L, a TRS-B: S, a TRS-B: orf3a, a TRS-B: E, a TRS-B: M, a TRS-B: orf6, a TRS-B: orf7a, a TRS-B: orf8 and/or a TRS-B: N are/is defined by the sequence ACGAAC.

In some embodiments, the nucleic acid sequence comprises a sequence defined by SEQ. ID. 41 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 41.

In some embodiments, the nucleic acid sequence comprises a sequence defined by SEQ. ID. 42 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 42.

In some embodiments, the nucleic acid sequence comprises a sequence defined by SEQ. ID. 43 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 43.

In some embodiments, the nucleic acid sequence comprises a sequence defined by SEQ. ID. 44 or a sequence having at least 98,5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 44.

In some embodiments, the nucleotide acid sequence described herein, refers to the corresponding ribonucleic acid sequence.

ORF6 and ORF8 of SARS-CoV-2 inhibit the type I interferon signaling pathway (Li, J. Y., et al., 2020, Virus research, 286, 198074) and therefore impede an adequate immune response. Therefore, deletion or omission of the sequence of ORF6 and/or ORF8 of SARS-CoV-2 in the vector does not only limit the reproducibility of the encoded viral particles but also increases antigenicity thereof.

Accordingly, the invention is at least in part based on the discovery that the nucleotide acid sequence of the invention encodes virus particles or parts thereof with a surprising antigenicity and limited replication capabilities.

In some embodiments, the nucleic acid sequence of the invention is a vector or part of a vector.

The term “vector”, as used herein, refers to a nucleic acid molecule, capable of transferring or transporting itself and/or another nucleic acid molecule into a cell. The transferred nucleic acid is generally linked to, i.e., inserted into, the vector nucleic acid molecule. A vector may include sequences that direct autonomous replication in a cell, or may include sequences sufficient to allow integration into host cell DNA. In some embodiments, the vector described herein is a vector selected from the group of plasmids (e.g., DNA plasmids or RNA plasmids), shuttle vectors, transposons, cosmids, bacterial artificial chromosomes, and viral vectors.

In certain embodiments, the invention relates to the vector according to the invention, wherein the vector does not comprise sequence part B and the regulation of sequence part A does not comprise at least one accessory protein.

In certain embodiments, the invention relates to the vector according to the invention, wherein the vector is a plasm id vector.

In some embodiments, the plasmid vector described herein has a selection marker and sequence determining the origin of replication. In some embodiments, the invention relates to a vector according to the invention, wherein the vector comprises the sequences defined by the SEQ. ID. 46 and SEQ. ID. 47.

In some embodiments, the invention relates to a vector according to the invention, wherein the vector comprises at least one sequence encoding an RNA-polymerase promoter and at least one untranslated region that contains sequences that enable the synthesis of negative-strand RNA and/or that enable positive-strand RNA synthesis.

In some embodiments, the invention relates to a vector according to the invention, wherein the vector comprises at least one sequence encoding an T7 promoter and at least two untranslated regions that contain sequences that enable the synthesis of negative-strand RNA and/or that enable positive-strand RNA synthesis.

In some embodiments, the invention relates to a vector according to the invention, wherein the vector comprises at least one sequence encoding an T7 promoter as defined by the SEQ. ID. 28, and at least two untranslated regions that comprise the sequences according to the SEQ. ID. 56 and 57.

In some embodiments, the invention relates to the vector according to the invention, wherein the vector is a plasm id vector.

In some embodiments, the invention relates to the vector according to the invention, wherein the vector comprises a sequence as defined in SEQ. ID. 45.

In some embodiments, the nucleotide acid sequence described herein, comprises a sequence having at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the SEQ. ID. 45.

In some embodiments, the vector described herein, comprises a sequence i) having at least 50%, at least 60%, at least 70%, at least 80%, at least 90%, at least 91%, at least 92%, at least 93%, at least 94%, at least 95%, at least 96%, at least 97%, at least 98%, or at least 99% sequence identity to the SEQ. ID. 45; and ii) comprising selection marker as defined by the SEQ. ID. 47 and an origin of replication as defined by the SEQ. ID. 46.

In some embodiments, the vector described herein is used in combination with at least one transfection enhancer, e.g., a transfection enhancer selected from the group of oligonucleotides, lipoplexes, polymersomes, polyplexes, dendrimers, inorganic nanoparticles and cell-penetrating peptides.

The vector described herein can be used for efficient transfer and/or amplification of the nucleic acid sequence of the invention in an amplifying biotechnological production unit (Example 3).

The product of the amplification in an amplifying biotechnological production unit (e.g., yeast cells) may be isolated and subsequently translated in a further biotechnological production unit (e.g., human cell).

Accordingly, the invention is at least in part based on the discovery that the vector described herein enables efficient amplification of the nucleic acids described herein and efficient production of a combination virus-like proteins with limited replication capabilities but high antigenicity. The inventive nucleic acids lead through the above procedure to the production of a dispersion comprising proteins and other building blocks.

Suitable separation methods known to the person skilled in the art, such as centrifugation or chromatography, can be used to separate these building blocks, if necessary also from residues of the production cell line used or other production aids or organisms, and thus purify them.

In some embodiments, the building blocks described herein are purified using at least one separation method selected from the group of chromatography, precipitation, ultracentrifugation, tangential-flow filtration, and enzymatic digestion

These optionally purified virus envelopes or fragments thereof represent the basis of the vaccine, which is then transferred into different dosage forms depending on the type of application.

Typically, an adjuvant is used for this purpose, stabilizers to improve shelf-life, salts and buffers. The vaccines are thus the product of the long-chain, fully synthetic nucleic acids described here.

In some embodiments, the invention relates to a virus envelope, a fragment of a virus envelope and/or virus envelope protein obtainable by gene expression using at least one nucleic acid according to the invention using the vector according to the invention, using the kit according to the invention, or the biotechnological production unit according to the invention, wherein the virus envelope, the fragment of a virus envelope and/or the virus envelope protein package the at least one nucleic acid according to the invention.

In some embodiments, the invention relates to a virus envelope obtainable by gene expression using at least one nucleic acid according to the invention using the vector according to the invention, using the kit according to the invention, or the biotechnological production unit according to the invention.

The term “virus envelope”, as used herein, refers to protein assembly such as a protein layer that has a stabilizing function for a nucleotide acid sequence (such as the nucleotide acid sequence of the invention). In some embodiments, the virus envelope described herein enables the assimilation of the nucleotide acid sequence of the invention into a human cell. In some embodiments, the virus envelope described herein comprises a spike protein, envelope protein and a membrane protein.

In some embodiments, the invention relates to a fragment of a virus envelope obtainable by gene expression using at least one nucleic acid according to the invention using the vector according to the invention, using the kit according to the invention, or the biotechnological production unit according to the invention.

The term “fragment of a virus envelope”, as used herein, refers to at least two assembled proteins that form an incomplete virus envelope.

In some embodiments, the invention relates to a virus envelope protein obtainable by gene expression using at least one nucleic acid according to the invention using the vector according to the invention, using the kit according to the invention, or the biotechnological production unit according to the invention.

The term “virus envelope protein”, as used herein, refers to at least one protein that can form part of a viral envelope.

In some embodiments, the invention relates to a virus envelope, a fragment of a virus envelope and/or virus envelope protein obtainable by gene expression using at least one nucleic acid according to the invention using the vector according to the invention, using the kit according to the invention, or the biotechnological production unit according to the invention, wherein the virus envelope, the fragment of a virus envelope and/or the virus envelope protein package the at least one nucleic acid according to the invention.

The term “packaged”, as used herein, refers to at least partially engulfed and/or linked. In some embodiments, the packaging nucleotide acid of the invention in the virus envelope, the fragment of a virus envelope and/or the virus envelope protein enables entrance into human cells.

The products of the nucleic acid and/or the vector of the invention show a particularly high antigenic similarity to the corresponding functional virus, if the products are embodied in a virus envelope, a fragment of a virus envelope and/or virus envelope protein. Therefore, the elicited/induced immune reaction will likely induce an immune reaction that is particularly beneficial for the actual contact with the functional virus.

The nucleotide acid packaged in the virus envelope, the fragment of a virus envelope and/or the virus envelope protein can be transferred into human cell of a subject and induce production of viral proteins in the human cell. This results in prolonged and enhanced exposure of antigenic virus-like proteins with limited replication capabilities.

Accordingly, the invention is at least in part based on the discovery that the vector described herein enables efficient production of a combination virus-like proteins with limited replication capabilities but similar antigenic effect to the original virus.

In some embodiments, the invention relates to the vector of the invention for use in treatment.

In some embodiments, the invention relates to the biotechnological production unit of the invention for use in treatment.

In some embodiments, the invention relates to the virus envelope, the fragment of a virus envelope and/or the virus envelope protein of the invention for use in treatment.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

In some embodiments, the invention relates the vector, the biotechnological production unit, the virus envelope, the fragment of a virus envelope and/or the virus envelope protein of the invention for use in the treatment of SARS-CoV-2 infections.

In some embodiments, the invention relates the vector, the biotechnological production unit, the virus envelope, the fragment of a virus envelope and/or the virus envelope protein of the invention for use in the prevention of SARS-CoV-2 infections.

In some embodiments, the invention relates the vector, the biotechnological production unit, the virus envelope, the fragment of a virus envelope and/or the virus envelope protein of the invention for use in the treatment of active SARS-CoV-2 infections.

In some embodiments, the invention relates to a vaccine against the coronavirus SARS-CoV-2 comprising at least one nucleic acid according to the invention and products obtainable by gene expression using at least one nucleic acid according to the invention in a production organism.

In some embodiments, the invention relates to a vaccine against the coronavirus SARS-CoV-2 comprising at least one nucleic acid according to the invention and products obtainable by using the vector according to the invention in a production organism.

In some embodiments, the invention relates to a vaccine against the coronavirus SARS-CoV-2 comprising at least one nucleic acid according to the invention and products obtainable by using the kit according to the invention in a production organism.

In some embodiments, the invention relates to a vaccine against the coronavirus SARS-CoV-2 comprising at least one nucleic acid according to the invention and products obtainable by gene expression using at least one nucleic acid according to the invention, using the vector according to the invention, using the kit according to the invention in a production organism, in particular comprising the virus envelope, the fragment of a virus envelope and/or the virus envelope protein according to the invention.

The term “vaccine”, as used herein, refers to any agent or composition, capable of inducing/eliciting an immune response in a host and which permits to treat and/or prevent an infection and/or a disease. Therefore, non-limiting examples of such agents include proteins, polypeptides, protein/polypeptide fragments, immunogens, antigens, peptide epitopes, epitopes, mixtures of proteins, peptides or epitopes as well as nucleic acids, genes and/or portions of genes (encoding a polypeptide or protein of interest or a fragment thereof).

The term “against the coronavirus SARS-CoV-2”, as used herein, refers to treatment and/or prevention of a SARS-CoV-2 infection. In some embodiments, the SARS-CoV-2 infection described herein is COVID-19.

The structural proteins of coronaviruses have shown to elicit an immune response (see, e.g., Li, J. Y., et al., 2020, Virus research, 286, 198074; Walls, A. C., et al., 2020, Cell, 181(2), 281-292.e6; Chen, Z, et al., 2004, Clinical chemistry, 50(6), 988-995; Peng, Y., et al., 2020, Nature immunology, 21(11), 1336-1345.). The means and methods provided enable to inducing/eliciting an equivalent immune response by the production and administration of a vaccine with the equivalent epitopes and/or particles with reduced immune evading mechanisms. In some embodiments, the vaccine induces production of particles with limited replicative capabilities in subject to

Thus, these vaccines thus differ massively from classical vaccines, which are often derived from animal serum and are therefore molecularly inconsistent. The production from animal organisms is traditionally the method of choice. However, the molecularly unclear products lead to massive quality problems and variation from production batch to production batch. This is also associated with the long approval period and the side effects that are often discovered only late. A molecularly defined product composition, as it can be obtained using the nucleic acid according to the invention, is therefore advantageous.

Furthermore, the vaccine described herein, is both clearly defined and offers a broad range of antigenic epitopes. This results in the advantage that the vaccine has a low or no requirement for adjuvants that enhance the immune response. Such adjuvants that enhance the immune response are typically associated with side effects such as allergic reactions in some patients. Furthermore, the primary active components of the vaccine as described herein are protein-based and are therefore more thermostable compared to other vaccines (e.g., RNA vaccines). The vaccine of the invention is therefore easily transportable and storable due to its stability.

Accordingly, the invention is a least in part based on the discovery, that the vaccine as described herein is particularly useful against the coronavirus SARS-CoV-2.

In some embodiments, the invention relates to a kit comprising two or more nucleic acids according to the invention.

In some embodiments, the invention relates to a kit comprising at least two nucleic acids selected from the group of SEQ. ID. 35, SEQ. ID. 36, SEQ. ID. 37, and SEQ. ID. 38.

In this combination of vectors, the kit enables production of viral proteins in a human cell.

In addition to the said nucleic acid, the invention also relates to a kit comprising two or more nucleic acids, wherein the nucleic acids are deoxyribonucleic acids (DNA) according to one of the preceding claims and/or corresponding ribonucleic acids (RNA) with the corresponding base-pair sequence. In other words, the corresponding ribonucleic acid has sequence parts as defined above in which thymine (T) is replaced by uracil (U).

The kit described herein, can be prepared by collecting the necessary biotechnological production unit(s) and reagents. If the nucleic acids comprised in the kit are present in the form of DNA, it is further preferred that they are present in at least one plasmid, preferably in two or more plasmids. This allows the nucleic acid to be easily introduced into a corresponding biotechnological production unit, as is also described in the context of the concrete examples below.

In a particularly embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

Accordingly, the invention is at least in part based on the discovery, that it enables efficient and safe production of virus particles and/or parts thereof.

According to another aspect, the invention thus also concerns a biotechnological production unit comprising at least one plasm id as defined above, in particular two or more plasmids. The production unit on which this further aspect of the invention is based is usually a production organism or cell line known to the skilled person for the purposes described

According to another aspect, the present invention also concerns the product resulting from the application of the corresponding long-chain, fully synthetic nucleic acids in a suitable production organism or cell line. These products belong to the class of envelope proteins, often with additional sugar or fatty acid groups. In concrete terms, this further aspect thus concerns a virus envelope, a fragment of a virus envelope and/or a virus envelope protein obtainable by gene expression using the nucleic acids or using the kit as defined above.

It is important here that the assignment is mathematically unambiguous: A nucleic acid i produces a product i that is precisely dependent on it. A nucleic acid j that is even slightly different produces another product j that is also precisely dependent on it. The two relationships between product and nucleic acids are unambiguous and describable. Each type of product k can be assigned to a nucleic acid k. It is therefore justified to speak of a direct relationship between the nucleic acid and the product, i.e. the virus envelope or fragments thereof.

Wherever alternatives for individual separable features are presented here as “embodiments”, it is understood that such alternatives can be freely combined to form discrete embodiments of the invention disclosed herein.

It should be mentioned that the assembly of virus envelopes is carried out at different speeds and varies in cleanliness depending on the organism and type, so in practice, envelopes and fragments thereof are always found together. If necessary, however, they can be separated by common methods.

In some embodiments, the envelopes described herein are purified using at least one purification method selected from the group of chromatography, precipitation, ultracentrifugation, tangential-flow filtration, and enzymatic digestion.

According to a further aspect of the invention, the direct product of the inventive long-chain nucleic acids is thus converted into a vaccine by means of optional purification steps and possible auxiliary means. Specifically, this further aspect thus concerns a vaccine comprising products obtainable by gene expression using at least one nucleic acid or kit as defined above in a production organism, in particular comprising one or more of the above-mentioned protein components or parts thereof.

This vaccine is typically a physiological saline solution with the above-mentioned additives and typically small concentrations of the above-described virus envelopes and/or fragments.

Although the vaccine described herein, is less dependent on the effect of adjuvants than other vaccines, the vaccine may still comprise adjuvants to enhance the effect of the vaccine. In some embodiments, the vaccine comprises at least one adjuvant selected from the group of inorganic compounds (e.g., potassium alum, aluminum hydroxide, aluminum phosphate, calcium phosphate hydroxide), oils (e.g., paraffin oil, peanut oil), bacterial products, saponins, cytokines (e.g., IL-1, IL-2, IL-12) and squalene.

In some embodiments, the vaccine is administered by at least one route of administration selected from the group of oral administration, rectal administration, inhalation, nasal administration, parental administration, intramuscular administration, subcutaneous administration and intradermal administration.

Typical vaccines are injected or can be applied through the mucous membranes, depending on the dosage form.

As mentioned above, the vaccine is specifically a vaccine against the coronavirus SARS-CoV-2. Specifically, it comprises at least two molecularly precisely defined protein components selected from the group consisting of the protein components a, b1, b2, c1 or c2, d1 or d2, whereby
(i) the protein component a comprises the sequence as defined by SEQ. ID. 14 and SEQ. ID. 18 analogous to the S protein of SARS-CoV-2; and
(ii) the protein component b1 comprises the sequence set out in SEQ. ID. 6 and SEQ. ID. 21 analogous to the envelope protein E of SARS-CoV-2 and protein component b2 comprises the sequence according to SEQ. ID. 8 analogous to the envelope protein E of MHV59A or an equivalent protein; and
(iii) the protein component c1 comprises the sequence according to SEQ. ID. 10 and SEQ. ID. 22 analogous to the membrane protein M of SARS-CoV-2 and protein component (c2) the sequence according to SEQ. ID. 12 analogous to membrane protein M of MHV59A or an equivalent protein; and
(iv) the protein component d1 comprises the sequence according to SEQ. ID. 2 and SEQ. ID. 26 analogous to the nucleocapsid phosphoprotein N of SARS-CoV-2 and the protein component d2 comprises the sequence according to SEQ. ID. 4 analogous to the nucleocapsid phosphoprotein N of MHV59A or an equivalent protein.

It should be noted that the protein components a, b1, b2, c1, c2, d1 or d2 are similar but not identical to the corresponding naturally occurring analogues, which results from the fact that they are produced from synthetic nucleic acids which differ in sequence from the sequence of the corresponding natural nucleic acids.

The protein component a disclosed within this description comprises a sequence according to SEQ. ID. 14 and SEQ. ID. 18. In some embodiments, the protein component a comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 14.

In some embodiments, the protein component a comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 18.

The protein component b1 disclosed within this description comprises a sequence according to SEQ. ID. 6 and SEQ. ID. 21. In some embodiments, the protein component b1 comprises a sequence at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 6.

In some embodiments, the protein component b1 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 21.

The protein component b2 disclosed within this description comprises a sequence according to SEQ. ID. 8. In some embodiments, the protein component b2 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 8.

The protein component c1 disclosed within this description comprises a sequence according to SEQ. ID. 10 and SEQ. ID. 22. In some embodiments, the protein component c1 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 10.

In some embodiments, the protein component c1 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 22.

The protein component c2 disclosed within this description comprises a sequence according to SEQ. ID. 12. In some embodiments, the protein component c2 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 12.

The protein component d1 disclosed within this description comprises a sequence according to SEQ. ID. 2 and SEQ. ID. 26. In some embodiments, the protein component d1 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 2.

In some embodiments, the protein component d1 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 26.

The protein component d2 disclosed within this description comprises a sequence according to SEQ. ID. 4. In some embodiments, the protein component d2 comprises a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 4.

A protein component with a certain % sequence identity to an amino acid sequence described herein can be obtained for example by insertion, deletion, substitution and/or modification of at least one amino acid, but not more than 10%, not more than 9%, not more than 8%, not more than 7%, not more than 6%, not more than 5%, not more than 4%, not more than 3%, not more than 2%, not more than 1%, not more than 0.9%, not more than 0.8%, not more than 0.7%, not more than 0.6%, not more than 0.5%, not more than 0.4%, not more than 0.3%, not more than 0.2% or not more than 0.1% of the amino acids relative to the amino acid sequence SEQ. ID. 2, SEQ. ID. 4, SEQ. ID. 6, SEQ. ID. 8, SEQ. ID. 10, SEQ. ID. 12, SEQ. ID. 14, SEQ. ID. 18, SEQ. ID. 21, SEQ. ID. 22, and/or SEQ. ID. 26. Such an insertion, deletion, substitution and/or modification can be achieved based on a corresponding nucleotide acid sequence described herein that encodes the desired insertion, deletion, substitution and/or modification (for example a nucleotide acid sequence of a SARS-CoV-2 variant encoding for a mutated variant of a protein component described herein).

The insertion, deletion, substitution and/or modification can also be the result of a post-translational modification. In some embodiments, the protein component described herein is post-translationally modified to improve the production process. In some embodiments, the protein component described herein is post-translationally modified to improve at least one protein property of the protein component described, such as a protein property selected from the group of antigenicity, protein stability, pharmacokinetic, pharmacodynamic, interactions with drugs and interactions with adjuvants. In some embodiments, the protein component described herein is post-translationally modified by at least technique selected from the group of addition of functional groups, linked to other proteins or peptides, chemical modification of amino acids (e.g., citrullination, deamination, deamidation, eliminylation), disulfide bridges, cysteine amino acid linkage, peptide bond cleavage, isoaspartate formation, racemization and protein splicing.

Thus, the amino acid sequences described herein do not necessarily have a proportional % sequence identity overlap as the nucleotide acid sequences described herein. In some embodiments, the amino acid sequence of the invention differs at least 10%, at least 9%, at least 8%, at least 7%, at least 6%, at least 5%, at least 4%, at least 3%, at least 2%, at least 1%, at least 0.9%, at least 0.8%, at least 0.7%, at least 0.6%, at least 0.5%, at least 0.4%, at least 0.3%, at least 0.2%, at least 0.1% more from the sequences described in SEQ. ID. 2, SEQ. ID. 4, SEQ. ID. 6, SEQ. ID. 8, SEQ. ID. 10, SEQ. ID. 12, SEQ. ID. 14, SEQ. ID. 18, SEQ. ID. 21, SEQ. ID. 22, and/or SEQ. ID. 26 than the altered nucleotide acid sequences differ from the nucleotide acid sequences described herein.

In some embodiments, the invention relates to the vaccine according to the invention comprising at least two molecularly precisely defined protein components selected from the group consisting of the protein components a, b1, b2, c1 or c2, d1 or d2, wherein
(i) the protein component a comprises
   a) the sequence according to SEQ. ID. 14 analogous to the S protein of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 14; or
   b) the sequence according to SEQ. ID. 18 analogous to the S protein of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.18;
(ii) the protein component b1 comprises
   a) the sequence according to SEQ. ID. 6 analogous to the envelope protein E of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.6; or
   b) the sequence according to SEQ. ID. 21 analogous to the envelope protein E of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.21; and
the protein component b2 comprises the sequence according to SEQ. ID. 8 analogous to the envelope protein E of MHV59A or an equivalent protein comprising a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.8;
(iii) the protein component c1 comprises
   a) the sequence according to SEQ. ID. 10 analogous to the envelope protein E of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 10; or
   b) the sequence according to SEQ. ID. 22 analogous to the membrane protein M of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.22; and
the protein component c2 comprises the sequence according to SEQ. ID. 12 analogous to membrane protein M of MHV59A or an equivalent protein comprising a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID.12; and
(iv) the protein component d1 comprises
   a) the sequence according to SEQ. ID. 2 analogous to the nucleocapsid phosphoprotein N of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 2; or
   b) the sequence according to SEQ. ID. 26 analogous to the nucleocapsid phosphoprotein N of SARS-CoV-2 or a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 26; and
the protein component d2 the sequence according to SEQ. ID. 4 analogous to the nucleocapsid phosphoprotein N of MHV59A or an equivalent protein comprising a sequence having at least 90%, having at least 91%, having at least 92%, having at least 93%, having at least 94%, having at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ. ID. 4.

In some embodiments, the invention relates to the vaccine according to the invention comprising at least two molecularly precisely defined protein components selected from the group consisting of the protein components a, b1, c1, and d1.

In some embodiments, the invention relates to the vaccine according to the invention comprising at least two molecularly precisely defined protein components selected from the group consisting of the protein components b1, c1, and d1.

In some embodiments, the invention relates to the vaccine according to the invention comprising at least two molecularly precisely defined protein components selected from the group consisting of the protein components a, c1, and d1.

In some embodiments, the invention relates to the vaccine according to the invention comprising at least two molecularly precisely defined protein components selected from the group consisting of the protein components a, b1, and d1.

In some embodiments, the invention relates to the vaccine according to the invention comprising at least two molecularly precisely defined protein components selected from the group consisting of the protein components a, b1, and c1

In some embodiments, the invention relates to the vaccine according to the invention comprising at least two molecularly precisely defined protein components a and c1

In some embodiments, the invention relates to the vaccine according to the invention comprising at least two molecularly precisely defined protein components a and d1.

In some embodiments, the invention relates to the vaccine according to the invention comprising at least two molecularly precisely defined protein components c1 and d1.

In some embodiments, the invention relates to the vaccine according to the invention comprising at least two molecularly precisely defined protein components a, and b1, c1, and d1.

In some embodiments, the invention relates to the vaccine according to the invention comprising at least three molecularly precisely defined protein components selected from the group consisting of the protein components a, b1, c1, and d1.

In some embodiments, the invention relates to the vaccine according to the invention comprising three molecularly precisely defined protein components selected from the group consisting of the protein components a, b1, c1, and d1.

The vaccine according to the invention comprising the protein components described herein can elicit a substantial and broad immune response. At the same time, the vaccine may be limited in replication capabilities, in that it does not replicate in the body of a subject. Such limited replication capabilities can for example be achieved by omitting or altering required sequences for efficient replication.

Accordingly, the invention is at least in part based on the discovery that a vaccine comprising the combination of protein components described herein, can show desired limitations in replication capabilities, while largely maintaining the antigenic potential.

Furthermore, the invention relates to a method for producing the vaccine comprising the successive steps of introducing at least one nucleic acid according to one of claims 1 to 10 into a biotechnological production unit, in particular a cell line, by means of transfection, starting from the nucleic acid-based mRNA, at least two of the protein components selected from the group consisting of the protein components a, b1, b2, c1, c2, d1 or d2 are prepared by translation and the protein components obtained thereof are purified.

In some embodiments, the invention relates to a method for the production of the vaccine according to the invention comprising the successive steps of
a) introducing the vector according to one of embodiments 10 to 14 into a biotechnological production unit, in particular a cell line,
wherein, the nucleic acid-based mRNA coding for at least two of the protein components selected from the group consisting of the protein components a, b1, b2, c1, c2, d1 or d2 are prepared by translation;
b) obtaining protein components from the biotechnological production unit in step a); and
c) purifying the obtained protein components to obtain the vaccine according to the invention.

In some embodiments, the invention relates to a biotechnological production unit comprising at least one vector according to the invention.

The terms “biotechnological production unit” and “production organism” are used herein interchangeably and refer to at least one host cell into which the nucleic acid of the invention has been introduced for expression, including the progeny of such cells, organisms and biotechnological units that comprise such cells and/or progeny of such cells. Host cells include “transformants” and “transformed cells,” which include the primary transformed cell and progeny derived therefrom without regard to the number of passages. The progeny may not be completely identical in nucleic acid content to a parent cell but may contain mutations. Mutant progeny that have the same function or biological activity as screened or selected for in the originally transformed cell are included herein.

The term “amplifying biotechnological production unit” refers to any biotechnological production unit that allows amplification of large vectors (e.g., more than 4000 bases, more than 10000 bases, more than 35000 bases). In some embodiments, the amplifying biotechnological production unit described herein comprises a yeast cell.

In certain embodiments, the host cell is a stem cell. In other embodiments, the host cell is a differentiated cell.

The biotechnological production unit described herein is particularly useful, if it comprises a cell that allows viral entry of SARS-CoV-2, such that the product of cells of the biotechnological production unit can enter further cells of the biotechnological production unit. This subsequent infection of cells of the biotechnological production facilitates and accelerates the process of bringing the vector into the host cells.

In some embodiments, the biotechnological production unit described herein comprises a cell that allows viral entry of SARS-CoV-2. In some embodiments, the biotechnological production unit described herein comprises a cell that expresses the human ACE2 receptor or a functional human-like ACE2 receptor. The human-like ACE2 receptors that allow viral entry of SARS-CoV-2 are known to the person skilled in the art (see, e.g., Damas, J., et al., 2020, Proceedings of the National Academy of Sciences, 117(36), 22311-22322).

In some the biotechnological production unit described herein comprises at least one cell type selected from the group of HEK293, MDCK, Chinese hamster ovary (CHO), SF9, Vero, MRC 5, Per.C6, PMK, and WI-38.

In some embodiments, the biotechnological production unit described herein comprises a cell that is at least partially human or a cell of an at least partially human cell line.

In some embodiments, the biotechnological production unit described herein comprises a cell that allows the production of a viral particle comprising the nucleotide of the invention or the vector of the invention that is selectively replicable in it is fully replicable in cells of the biotechnological production unit but not or unsubstantially in cells of the human body. This selective replicability is achieved by cells that comprise complementary proteins for the replication of the viral particle (see e.g. Example).

In some embodiments, the biotechnological production unit described herein comprises a cell that can express at least one protein for viral replication. In some embodiments, the biotechnological production unit described herein comprises a cell that can express at least one protein component for viral replication that is not encoded in the nucleotide acid sequence of the invention or the vector of the invention.

Transduction of host cells by the vector of the invention can be achieved by stable or transient transduction (see, e.g., Stepanenko, A. A., and Heng, H. H., 2017, Mutation Research/Reviews in Mutation Research, 773, 91-103).

If DNA is introduced into the production unit according to a first embodiment, this is usually done using a plasm id suitable for this purpose.

Alternatively, the DNA may be introduced into the biotechnological production unit by any kind of vector.

If, on the other hand, RNA is introduced according to a second embodiment, a sequence coding for the RNA-dependent RNA polymerase (according to SEQ. ID. 30) is introduced in addition to the sequences coding for the protein components a, b1, b2, c1, c2, d1 or d2. This sequence makes it possible to first form a negative RNA strand from the positive RNA strand present as a template and then to produce the corresponding messenger RNA from it.

In the context of this second embodiment of the procedure, it is preferred that the vaccine obtained additionally comprises a fully synthetic, long-chain ribonucleic acid (according to SEQ. ID. 33 or 34), which is obtainable by enzymatic transcription.

In the context of this second embodiment of the procedure, it is also preferred that the vaccine obtained additionally comprises a fully synthetic, long-chain ribonucleic acid (according to SEQ. ID. 33 or 34), which is obtainable via the T7 transcription of the sequence according to SEQ. ID. 28.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives. Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The invention is further illustrated by the following design examples in combination with the attached figures, which do not limit the scope of the invention described in the claims.

### DESCRIPTION OF THE FIGURES

FIG. 1: Plasmid maps of the mono-cistronic expression plasm ids coding for the nucleocapsid protein (N) (SEQ. ID. 35), envelope protein (E) (SEQ. ID. 36), membrane protein (M) (SEQ. ID. 37) and spike glycoprotein (S) (SEQ. ID. 38) of SARS-CoV2. The numbers on the inside the plasmid maps indicate the DNA coordinates in base-pairs. The protein-coding sequences of N, E, M and S are represented by arrows and stand for the DNA and protein sequences with the SEQ. ID. 1,2,3 and 4 (N), 5,6,7 and 8 (E), 9, 10, 11 and 12 (M), 13 and 14 (S) as set forth in the Sequence Listing.

FIG. 2: Genome map of the poly-cistronic expression construct COVAX191AN (SEQ. ID. 33 and 39) (upper figure) which, together with the mono-cistronic expression plasmid pcDNA34 syn N (SEQ. ID. 35) (lower figure) can be used for vaccine production in cell lines as shown in example 2. The numbers refer to the DNA coordinates in kilobases (K) for COVAX191AN and refer to the base-pair positions for the pcDNA34 syn N construct (SEQ. ID. 35). Protein-coding sequences of the polyprotein 1a and 1 b, E, M S (upper figure) and the nucleocapsid protein syn N (lower figure) are represented by arrows.

FIG. 3: Agarose gel electrophoresis size separation of the mono-cistronic, plasmid-based expression constructs for the nucleocapsid protein (N), envelope protein (E), membrane protein (M) and the spike glycoprotein (S). The left side of the gel shows the MHV A59 (MHV) derived constructs for the nucleocapsid protein (N), envelope protein (E) and the membrane protein (M). The right side of the gel shows the derived constructs based on the SARS-CoV2 for the nucleocapsid protein (N), envelope protein (E), membrane protein (M) and the spike glycoprotein (S).

FIG. 4: Schematic illustration with the corresponding DNA sequencing cover graph of the circular 40,556 bp DNA construct COVAX191AN (SEQ. ID. 40) (upper figure) and the 38,383 bp DNA construct COVAX191ANAHE (SEQ. ID. 40) (lower figure). The arrows indicate the positions of the protein-coding sequences for the recoded CDS of the replicative polyproteins 1A and 1B (1A,1B), the hemagglutinin esterase (HE), the spike glycoprotein (S), the envelope protein (E) and the membrane protein (M). The complete genomes of COVAX191AN and COVAX191ANAHE were assembled from 6 synthetic DNA blocks using a single lithium acetate yeast transformation and selected for the auxotrophic URA3 marker.

FIG. 5: Schematic illustration of the SARS-CoV-2 genome and the deletion variants generated.

FIG. 6: Vector map of pcDNA3.1/Hygro(+)_ORF7a for trans-complementary expression of ORF7a (SEQ. ID. 61)

Table 51: DNA assembly efficiency of COVAX191 in S. cerevisiae (yeast)

### EXAMPLES

The following examples illustrate how the inventive long-chain nucleic acids encoding for the envelope proteins E, M, N and S are produced and used in a biotechnological process to stimulate cells to produce corona virus envelopes or fragments thereof.

For the production, the (digital) sequences according to the present invention are transferred into the corresponding physically present long-chain fully synthetic nucleic acid molecules by the process of chemical DNA synthesis.

### Example 1

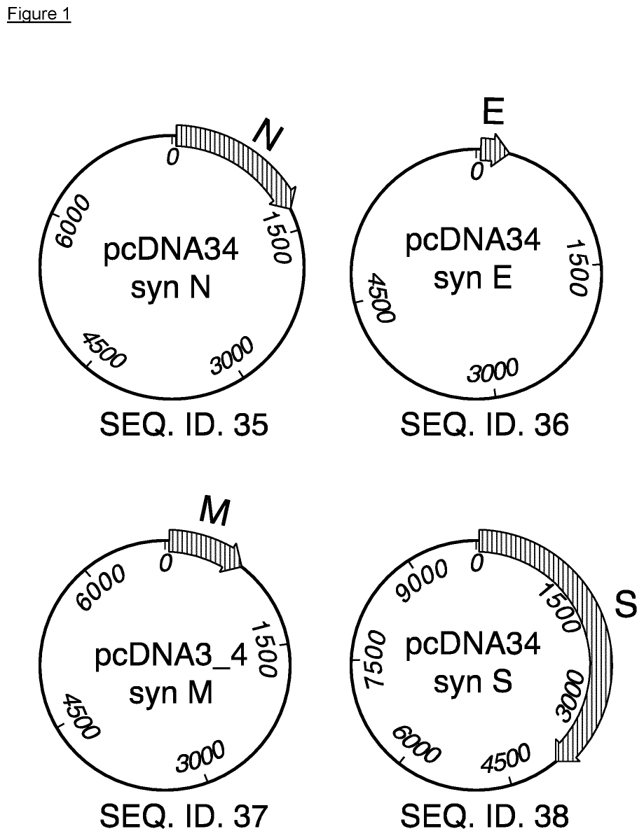

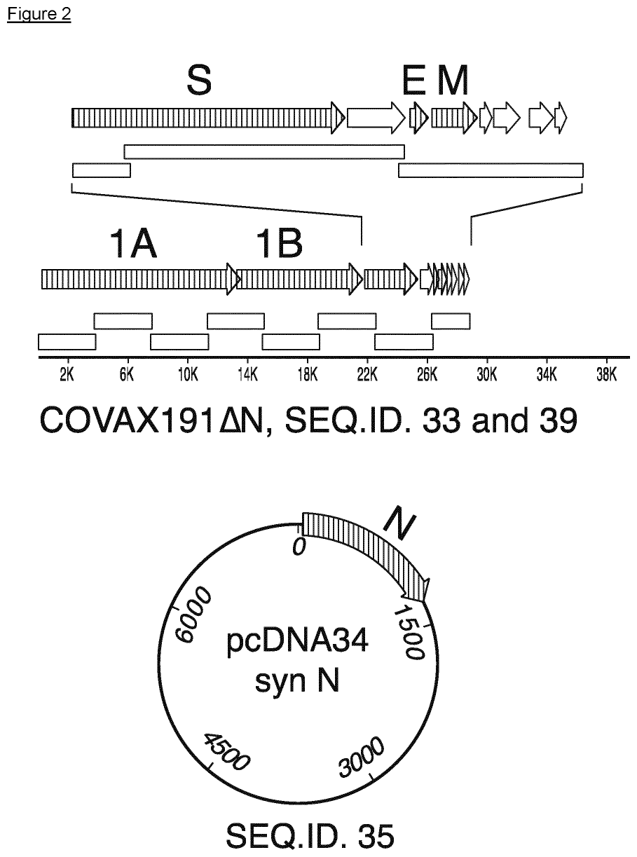

In the first example, the resulting long-chain, fully synthetic nucleic acids encoding the envelope proteins E, M, N and S are mono-cistronic, i.e. they are produced under the control of a separate promoter (SV40, CMV, EF-1, chicken 13 actin promoter or hybrid promoters) and other optional translation initiation signals (Kozak consensus sequence) and nuclear mRNA export signals (Chuck Wood sequence) into expression plasmids for eukaryotic cells. The sequences as revealed in SEQ. ID. 35, SEQ. ID. 36, SEQ. ID. 37 and SEQ. ID. 38, and FIG. 1 shall serve as an example of such an expression system. Other embodiments with other expression plasmids, the corresponding resistance genes and promoters are possible and are known to the person skilled in the art.

The resulting 4 expression plasmids are amplified in Escherichia coli, purified by standard chemical-physical procedures and then introduced by transfection into a eukaryotic cell line (HEK293, Chinese hamster ovary (CHO), SF9, Vero). Transfection is performed by standard procedures such as calcium phosphates, lipofection, electroporation.

After transfection, the cells, starting from the transfected plasmid DNA, begin to translate the messenger RNA (mRNA), from which the envelope proteins E, M, N and S are expressed by translation. These proteins spontaneously assemble in the cells to form corona virus envelopes and are then released by the cells by exocytosis into the culture medium, where they accumulate after 5-7 days.

Chemical-physical processes are used for the purification of envelope proteins, virus envelopes and their fragments. For this purpose, the cell culture supernatant is separated from the cells by centrifugation. In the subsequent step, the virus envelopes are further purified from impurities and other components of the culture medium by chromatographic column separation methods. The material thus obtained in its pure form, consisting of the coronavirus envelopes, forms the basis of the vaccine, which is then converted into various forms for administration depending on the type of application. Typically, an adjuvant is used for this purpose, stabilizers to improve the shelf-life, salts and buffers. The vaccines are thus the product of the long-chain, fully synthetic nucleic acids described here.

### Example 2

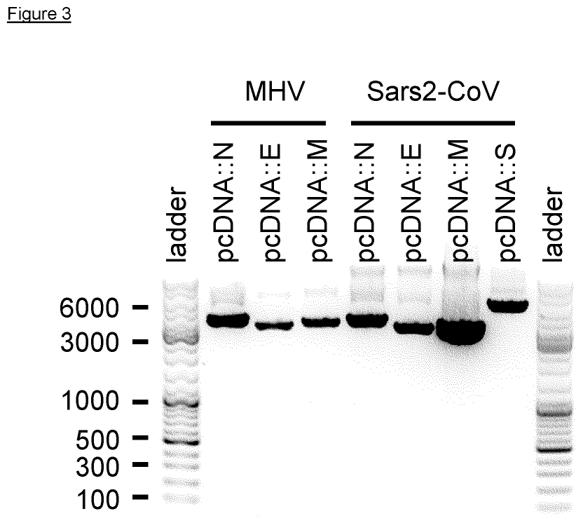

In the second example, the long-chain, fully synthetic nucleic acids encoding the envelope proteins E, M and S are expressed together with a fully synthetic nucleic acid encoding the RNA-dependent RNA polymerase. In this poly-cistronic expression system as revealed by the sequences SEQ. ID. 39 and SEQ. ID. 40 and shown in FIG. 2, the envelope proteins E, M and S are directly transcribed from a negative RNA strand, including the RNA-dependent RNA polymerase. If not all classes of envelope proteins of the sequence groups A-D are expressed RNA-dependent, additional expression plasm ids as described in example 1 can be used to express the complete set of envelope proteins for the biotechnological production of virus envelopes in cell lines. In example 2, the expression plasm id coding for the N protein is used for this purpose (SEQ. ID. 35) (see FIG. 2).

The purification of the plasmids, the transfection of the long-chain nucleic acids, as well as the purification of the virus envelopes, largely follows the process sequence described in example 1. However, the process includes an additional step in which the long-chain nucleic acid, as described in SEQ. ID. 39 and SEQ. ID. 40, is transformed by a T7 RNA polymerase into the corresponding RNA form according to SEQ. ID. 33 and SEQ. ID. 34 before transfection. This positive RNA strand leads to the production of the RNA-dependent RNA polymerase in the cell line, which produces a negative RNA strand from it. Transcription of the messenger RNA (mRNA) from this negative RNA strand then takes place, which leads to the production and assembly of the envelope proteins in virus envelopes.

The vaccine produced in this way differs from the vaccine described in the first example 1 in that, in addition to the envelope proteins obtained through the gene expression of the corresponding deoxyribonucleic acid, it contains a fully synthetic, long-chain ribonucleic acid which is expressed via the T7 transcription of the sequences SEQ. ID. 39 and SEQ. ID. 40.

The second example 2 has the advantage over the first application example that it produces virus envelopes that multiply themselves in a helper cell line expressing the

N protein. This is possible because the virus envelopes formed in this way additionally contain a positive RNA strand that codes for the RNA-dependent RNA polymerase and the envelope proteins E, M and S. If these virus envelopes are taken up by a cell, the cell itself is stimulated to produce virus envelopes. If the cell expresses the N protein episomally, which is the case for the vaccine production cell line, self-replicating virus envelopes are formed. This simplifies the production process and can be done without expensive transfection reagents. If the target cell does not express any N-protein, virus envelopes are also formed from it, but then they are devoid of a packaged RNA strand and can no longer self-replicate. These virus envelopes have the same chemical/physical structure and the same antigenicity as virus envelopes produced by the manufacturing process shown in example 1. Example 2 allows the production of virus envelopes, fragments and virus envelope proteins in further helper cell lines and production organisms as well as the direct application as RNA vaccine.

Methods:

Cultivation of Bacteria and Yeast Strains

Escherichia coli (E. coli) DH5alpha was cultivated in Luria-Broth (LB) at 37° C. Saccharomyces cerevisiae VL6-48N (Kouprina et al. 2006 Methods in Mol. Biol. 349, 85-101) was cultivated either in yeast peptone-dextrose (YPD) medium or synthetic dropout (SD) medium without uracil at 30° C.

Sequence design and de-novo DNA synthesis.

DNA sequences for mono-cistronic and poly-cistronic expression constructs were assembled from sequence parts disclosed in the attached sequence list (SEQ. ID. 1 to 40). Synthesis restrictions were removed computationally by synonymous codon replacement and application of the desired base substitutions within intergenic sequences. To define the optimal retro-synthetic assembly route, the synthesis-optimized DNA designs were hierarchically divided into smaller DNA fragments suitable for low-cost synthesis by commercial suppliers. The partitioning strategy was designed as a four-step, hierarchical assembly process. Sub-blocks with a size of 1.4 kb (kilobases) were assembled to blocks of 5.4 kb and further assembled to segments with a size of 16 kb and then into the final COVAX constructs of 35 to 40 kb. The linear DNA assembly parts have homology overlaps at the ends and nested 3′ prefix and 5′ suffix sequences to integrate assembled DNA parts into vectors and allow hierarchical assembly of the final COVAX DNA designs. The DNA assembly parts were obtained from commercial suppliers by low-cost DNA synthesis as sequence-verified, clonal plasmid constructs and double-stranded linear DNA.

Production of the mono-cistronic expression constructs:

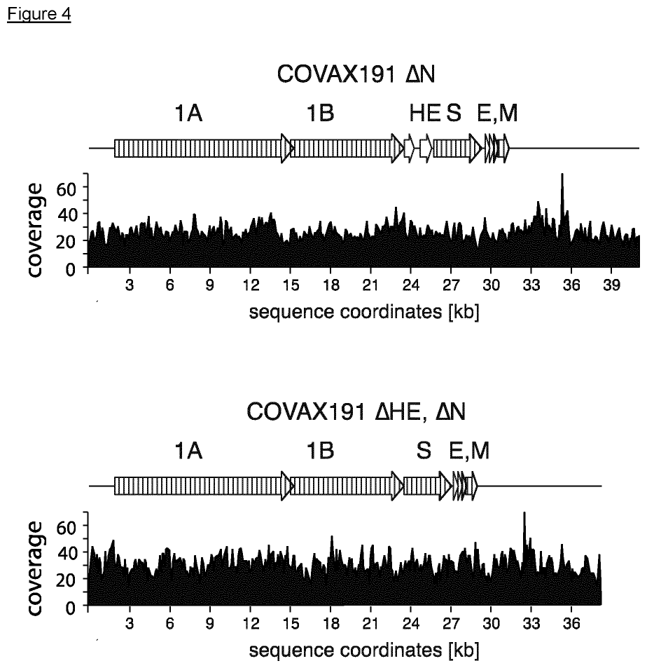

The synthetic nucleic acid sequences covering the complete protein-coding sequences of the S-protein of SARS-2 CoV, the M-protein, the N-protein and the E-protein of SARS-CoV-2 or MHV were amplified by polymerase amplification techniques (PCR) from sequence-verified synthetic DNA. Translation initiation sites before the start codon were introduced by oligonucleotide primers. The PCR products were separated by agarose gel electrophoresis according to their molecular weight and then purified over a nucleospin column (NucleoSpin Gel and PCR Clean-up Kit, Macherey nail). PCR products were cloned into the pcDNA3.4 vector using the Topo-TA cloning kit (TOPO-TA cloning kit, ThermoFisher). The molecular weight of the plasmids was determined by agarose gel electrophoresis (FIG. 3) and the DNA sequence was checked by Sanger sequencing.

Production of the Poly-Cistronic COVAX DNA Constructs:

The DNA assembly parts for the poly-cistronic COVAX DNA constructs were released from plasmids by restriction digestion using the Type IIS restriction enzymes (Bbsl, BspQI, Pacl and Pmel (New England Biolabs)). Equimolar amounts of DNA inserts (100 ng, 0.115 μmol) and linearized vector pXMCS2 (100 ng, 0.038 μmol) were incubated together with T5 exonuclease, phusion polymerase and Taq DNA ligase for one hour at 50° C. After isothermal assembly, the constructs were electroporated into E. coli DH5alpha cells (BioRad MiniPulser). The cells were incubated in LB medium for one hour and then plated out on LB plates. Segments and complete COVAX construct were assembled from blocks by yeast recombination using the plasmid pMR10Y (pMR10::CEN/ARS::URA3, Christen et al. 2015, ACS Synthetic Biology, 4, 927-934) according to the lithium acetate transformation method (Gietz et al 2007, Nature Protocols, 2, 31-34). Saccharomyces cerevisiae VL6-48N was grown overnight in 5 ml YPD, diluted 1:20 in 50 ml YPD and incubated for 4 h. The cells were collected by centrifugation at 1,000 rcf for 5 min, washed with 25 ml distilled water and centrifuged at 3,000 rcf for 5 min. The pellet was dissolved in 1 ml lithium acetate mixture (0.1 M lithium acetate, 0.01 M Tris-HCl, pH 7.5, 0.001 M EDTA, pH 8.0). Next, 100 μl single-stranded salmon sperm DNA (1% w/v salmon sperm DNA (ssDNA), 0.01 M Tris-HCl, pH 7.5, 0.001 M EDTA, pH 8.0) and 6 ml PEG-mix (40% w/v poly (ethylene glycol) 3015-3685 g/mol, 0.01 M Tris-HCl, pH 7.5, 0.001 M EDTA, pH 8.0) were added. From the PEG cell mix, 710 μl aliquots were combined with 100 ng of digested DNA blocks and 250 ng of linearized pMR10Y vector (Pact, Pmel). The samples were incubated for 30 minutes at 30° C. After incubation, 70 μl dimethyl sulfoxide (DMSO) was added and the samples were subjected to heat shock at 42° C. for 15 minutes. The cells were collected by centrifugation at 1000 rcf for 2 minutes and then plated out on SD plates without uracil and cultivated at 30° C. for three days until colonies became visible (see table 51).

Sequence verification of the COVAX DNA constructs.

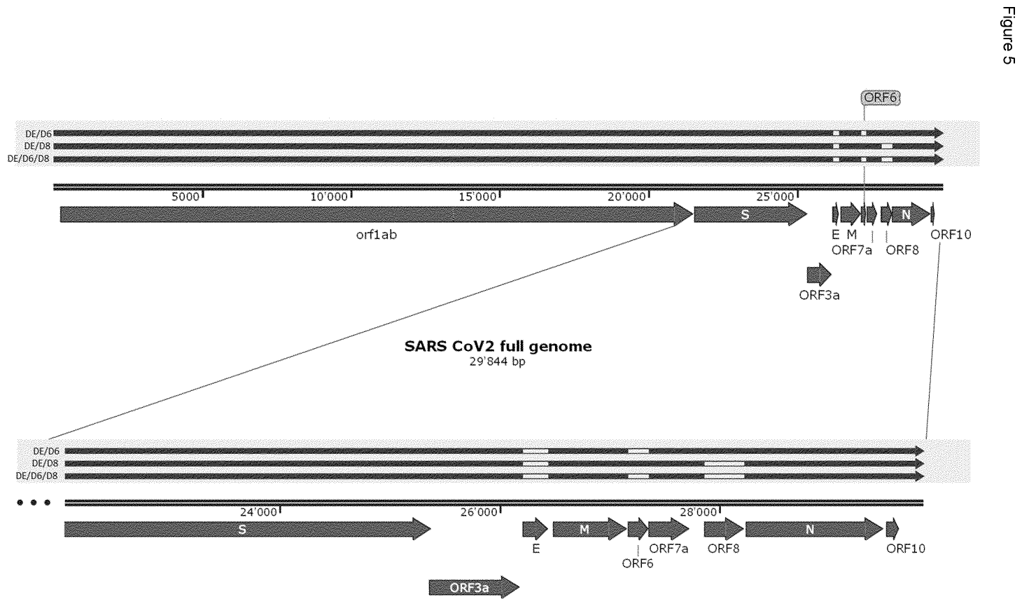

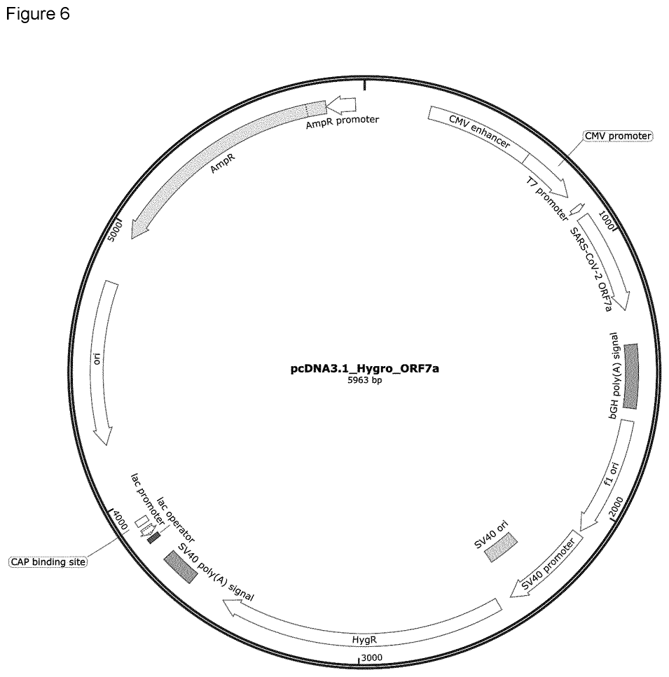

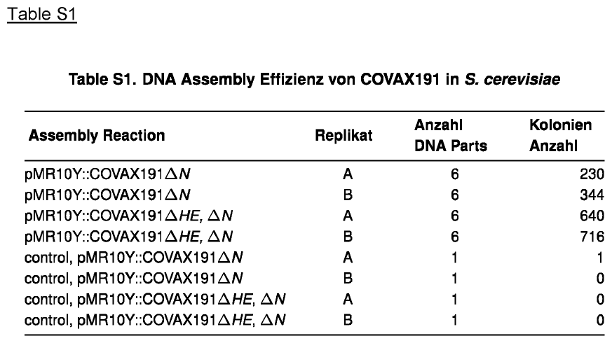

Sequence verification of the assembled DNA constructs was performed on an iSeq instrument (IIlumina) using the Nextera DNA Flex Library Prep-Kit. Genomic DNA of ura+yeast transformants was fragmented and processed according to the tagging protocol as specified by the manufacturer. Sequences were calculated de novo from the read sequences and the created contigs were compared with the reference sequences using the CLC Genomics Workbench software (Quiagen). The complete assembly of COVAX191AN and COVAX191AHEN was confirmed with a completely closed sequence coverage plot (FIG. 4).

### Example 3

Yeast clones each containing one of the circular sequences (viral sequences, T7 promoter and polyA-signal as well as vector, all together in one yeast artificial chromosome or “YAC”) were be grown, harvested and the YACs extracted thereof. The so-obtained YAC were cut with the restriction enzyme Eagl, leading to a double-stranded DNA molecule linearized directly after the polyA-signal. After making these DNA molecules RNase-free by standard treatment with Proteinase K followed by Trizol (phenol/chloroform) extraction, single-stranded RNA corresponding to the vaccine virus genome were obtained by in vitro transcription using T7 polymerase. The so obtained RNA were transfected into suitable cell lines (HEK293T or Vero cells). In the case of the positive control, the full-length construct “GBsyn_V33” unaltered HEK293 or Vero cells supported the replication of the RNA genome, the generation of subgenomic mRNAs and hence translation into viral proteins. These, together with the positive-strand RNA genome, and components from the cell membrane, formed progeny viruses, in this case wild-type, natural SARS-CoV-2 viruses. In the case of the deletion mutants, the gene or genes deleted in the virus genome are transfected into the cell lines in the form of DNA, leading to the transient expression of the protein or proteins, and thereby providing the missing factor required for enabling the generation of progeny virus. Alternatively (and preferred), cultivation of those cells under selection pressure leads to the stable integration of the gene or the genes into the cell genome, from where the protein or proteins are continuously expressed (with expression we understand the generation of mRNA from the gene and the subsequent translation into proteins). Such cells, either transiently or stably expressing the proteins made from the genes missing in the vaccine virus genome, enable a continuous production of vaccine viruses, characterized by a full set of structural proteins and a vaccine virus genome having one or several genes deleted. The so obtained vaccine viruses were purified in a so-called downstream processing (DSP) process characterized by clarification (separation of cells from the vaccine viruses), DNA digestion by Benzoase, Ultra Filtration/Dia Filtration (“UF/DF”) and finally sterile filtration (0.22 μm filtration).

### Example 4

De novo synthesis of a fully synthetic vector according to the methods described in Example 1 to 3 with an additional deletion or ORF7a: The de novo synthesis can use SEQ. ID. 60, SEQ. ID.41, SEQ. ID 42, SEQ. ID. 43, SEQ. ID. 44 as a reference sequence. Thereby, the functionality and/or expression of ORF7a encoding nucleotide sequence can be removed by a deletion and/or implementation of a dysfunctionality of the nucleotides 27388-27393 of SEQ. ID. 60, nucleotides 27000-27365 of SEQ. ID. 41, nucleotides 27196-27561 of SEQ. ID. 42, nucleotides 27000-27365 of SEQ. ID. 43 or nucleotides 27474-27839 of SEQ. ID. 44. Therefore a deletion of ORF7a alone or in combination with deletions E-protein, ORF6 and/or ORF8 may be achieved.

To facilitate the expression of such a vector a plasmid comprising SEQ. ID. 61 may be used for trans-complementary expression of the ORF7a.

1. Fully synthetic, long-chain nucleic acid with at least 4,000 bases, characterised in that the nucleic acid comprises
a) at least two of the four sequence parts A-D in any arrangement, wherein
i) Sequence part A comprises a sequence as defined in SEQ. ID. 50 or a sequence having at least 98.5% sequence identity to the sequence as defined in SEQ. ID. 50;
ii) Sequence part B comprises a sequence as defined in SEQ. ID. 48 or a sequence having at least 98.3% sequence identity to the sequence as defined in SEQ. ID. 48;
iii) Sequence part C comprises a sequence as defined in SEQ. ID. 49 or a sequence having at least 97.2% sequence identity to the sequence as defined in SEQ. ID. 49;
iv) Sequence part D comprises a sequence as defined in SEQ. ID. 17 or a sequence having at least 98.5% sequence identity to the sequence as defined in SEQ. ID. 17; or
encompasses a ribonucleic acid sequence corresponding to the deoxyribonucleic acid sequence according to the sequence parts A-D; and
b) 1.) a nucleic acid sequence part encodes an amino acid sequence having the function of a SARS-CoV-2 amino acid sequence encoded by ORF7a; and/or
2.) a nucleic acid sequence part encodes an amino acid sequence having the function of a SARS-CoV-2 amino acid sequence encoded by ORF3a.

2. The nucleic acid according to claim 1, characterized in that it has at least 8′000 bases, preferably at least 20′000 bases, in a defined sequence.

3. The nucleic acid according to claim 1 or 2, characterized in that the nucleic acid comprises not more than one or no ORF-associated nucleic acid sequence parts, wherein the ORF-associated nucleic acid sequence part encodes an amino acid sequence having the function of a SARS-CoV-2 amino acid sequence encoded by ORF6 or ORFS.

4. The nucleic acid according to claim 3, wherein the nucleic acid comprises no ORF-associated nucleic acid sequence part, wherein the ORF-associated nucleic acid sequence part encodes an amino acid sequence having the function of a SARS-CoV-2 amino acid sequence encoded by ORF6 or ORF8.

5. The nucleic acid according to one of the preceding claims, wherein the nucleic acid additionally comprises
a) 1.) an ORF1ab sequence defined by the SEQ. ID. 51 or a sequence having at least 98,5% sequence identity to SEQ. ID. 51; or
2. i) an ORF1b sequence defined by the SEQ. ID. 59 or a sequence having at least 98,5% sequence identity to SEQ. ID. 59; and
ii) a n ORF1a sequence defined by the SEQ. ID. 58 or a sequence having at least 98,6% sequence identity to SEQ. ID. 58; and
b) an ORF3a sequence defined by the SEQ. ID. 52 or a sequence having at least 99% sequence identity to SEQ. ID 52.

6. The nucleic acid according to claim 7, wherein the nucleic acid additionally comprises
a) an ORF6 sequence defined by the SEQ. ID. 53 or a sequence having at least 94,1% sequence identity to SEQ. ID 53; and/or
b) an ORF8 sequence defined by the SEQ. ID. 55 or a sequence having at least 99% sequence identity to SEQ. ID 55.

7. The nucleic acid according to one of the preceding claims, characterized in that sequence parts A to C correspond to the sequence according to SEQ. ID. 19 or the corresponding ribonucleic acid sequence.

8. The nucleic acid according to any of the preceding claims, characterized in that the nucleic acid comprises in any arrangement at least three of the four sequence parts A-D or at least three of four sequence parts with a ribonucleic acid sequence corresponding to the deoxyribonucleic acid sequence according to the sequence parts A-D.

9. The nucleic acid according to any of the preceding claims, characterized in that the nucleic acid comprises in any arrangement the four sequence parts A-D or four sequence parts with a ribonucleic acid sequence corresponding to the deoxyribonucleic acid sequence according to the sequence parts A-D.

10. The nucleic acid according to any one of claims 1 to 6, characterized in that the nucleic acid comprises two or three of the four sequence parts A-D.

11. The nucleic acid according to claim 10, characterized in that the nucleic acid comprises three of the four sequence parts A-D.

12. The nucleic acid according to one of the preceding claims, characterized in that the nucleic acid additionally comprises SEQ. ID. 28 and/or SEQ. ID. 29 or the corresponding ribonucleic acid sequence.

13. The nucleic acid according to one of the preceding claims, characterized in that it has a maximum size of 1′000′000 bases, preferably a maximum size of 200′000 bases.

14. A vector comprising the nucleic acid according to one of the preceding claims.

15. The vector according to claim 14, wherein the vector comprises the sequences defined by the SEQ. ID. 46 and SEQ. ID. 47.

16. The vector according to any one of the claims 14 to 15, wherein the vector is a plasm id vector.

17. A kit comprising two or more nucleic acids according to one of claims 1 to 13.

18. The kit according to claim 17, wherein the nucleic acids are present in at least one plasmid, preferably in two or more plasm ids.

19. A biotechnological production unit comprising at least one vector according to claims 14 to 16.

20. A virus envelope, a fragment of a virus envelope and/or virus envelope protein obtainable by gene expression using at least one nucleic acid according to one of claims 1 to 3, using the vector according to one of claims 14 to 16, using the kit according to one of claim 17 or 18, or the biotechnological production unit according to claim 19, wherein the virus envelope, the fragment of a virus envelope and/or the virus envelope protein package the at least one nucleic acid according to one of claims 1 to 13.

21. A vaccine against the coronavirus SARS-CoV-2 comprising at least one nucleic acid according to one of claims 1 to 13 and products obtainable by gene expression using at least one nucleic acid according to one of the claims 1 to 13, using the vector according to one of claims 14 to 16, using the kit according to one of the claim 17 or 18 in a production organism, in particular comprising the virus envelope, the fragment of a virus envelope and/or the virus envelope protein according to claim 20.

22. The vaccine according to claim 21 comprising at least two molecularly precisely defined protein components selected from the group consisting of the protein components a, b1, c1, or d1 wherein
(i) the protein component a comprises
a) the sequence according to SEQ. ID. 14 analogous to the S protein of SARS-CoV-2 or a sequence having at least 90% sequence identity to SEQ. ID. 14; or
b) the sequence according to SEQ. ID. 18 analogous to the S protein of SARS-CoV-2 or sequence having at least 90% sequence identity to SEQ. ID.18;
(ii) the protein component b1 comprises
a) the sequence according to SEQ. ID. 6 analogous to the envelope protein E of SARS-CoV-2 or a sequence having at least 90% sequence identity to SEQ. ID.6; or
b) the sequence according to SEQ. ID. 21 analogous to the envelope protein E of SARS-CoV-2 or a sequence having at least 90% sequence identity to SEQ. ID.21; and
(iii) the protein component c1 comprises
a) the sequence according to SEQ. ID. 10 analogous to the envelope protein M of SARS-CoV-2 or a sequence having at least 90% sequence identity to SEQ. ID. 10; or
b) the sequence according to SEQ. ID. 22 analogous to the membrane protein M of SARS-CoV-2 or a sequence having at least 90% sequence identity to SEQ. ID.22; and
(iv) the protein component d1 comprises
a) the sequence according to SEQ. ID. 2 analogous to the nucleocapsid phosphoprotein N of SARS-CoV-2 or a sequence having at least 90% sequence identity to SEQ. ID. 2; or
b) the sequence according to SEQ. ID. 26 analogous to the nucleocapsid phosphoprotein N of SARS-CoV-2 or a sequence having at least 90% sequence identity to SEQ. ID. 26.

23. A method for the production of the vaccine a vaccine against the coronavirus SARS-CoV-2 comprising the successive steps of a) introducing the nucleotide acid sequence according to one of claims 1 to 13 into a biotechnological production unit, in particular a cell line, wherein, the nucleic acid-based mRNA coding for at least two of the protein components selected from the group consisting of the protein components a, b1, b2, c1, c2, d1 or d2 are prepared by translation;
b) obtaining protein components from the biotechnological production unit in step a); and
c) purifying the obtained protein components to obtain the vaccine against the coronavirus SARS-CoV-2.

24. A method for the production of a vaccine against the coronavirus SARS-CoV-2 comprising the virus envelope, the fragment of a virus envelope and/or the virus envelope protein according to claim 20 comprising the successive steps of:
a) introducing the nucleotide acid sequence according to one of claims 1 to 13 into a biotechnological production unit, wherein the biotechnological production unit comprises a nucleotide acid coding for at least one of the protein components selected from the group consisting of the protein components a, b1, c1, and d1.
b) obtaining a fragment of a virus envelope and/or virus envelope protein from the biotechnological production unit in step a); and
c) purifying the obtained protein components to obtain the vaccine against the coronavirus SARS-CoV-2 comprising the virus envelope, the fragment of a virus envelope and/or the virus envelope protein according to claim 20.

25. A method for the production of a vaccine against the coronavirus SARS-CoV-2 comprising the successive steps of:
a) introducing the vector according to one of claims 14 to 16 into an amplifying biotechnological production unit;
b) amplifying the nucleotide acid according to one of claims 1 to 13 in the amplifying biotechnological production unit;
c) obtaining the nucleotide acid amplified in step b);
d) obtaining the vaccine against the coronavirus SARS-CoV-2 by using method according to claim 23 or 24.
